(19) United States
(12) Patent Application Publication
(10) Pub. No.: US 2024/0017255 A1
(43) Pub. Date: Jan. 18, 2024

(54) MVP CARTRIDGE AND METHODS OF USE AND MANUFACTURE

(71) Applicant: Cepheid, Sunnyvale, CA (US)

(72) Inventors: Rosa YU, Sunnyvale, CA (US); Robert Jones, Sunnyvale, CA (US); Jose Lopez, Sunnyvale, CA (US); Jun Wang, Sunnyvale, CA (US); Baback Bastin, Sunnyvale, CA (US)

(21) Appl. No.: 18/320,837

(22) Filed: May 19, 2023

Related U.S. Application Data
(60) Provisional application No. 63/343,969, filed on May 19, 2022.

(51) Int. Cl. B01L 3/00; C12Q 1/689; C12Q 1/6893; C12Q 1/6895; B01L 7/00

(52) U.S. Cl. CPC B01L 3/502738; C12Q 1/689; C12Q 1/6893; C12Q 1/6895; B01L 7/52; B01L 2200/16; B01L 2300/0681; B01L 2400/0644; C12Q 2600/16

(57) ABSTRACT

Test cartridges for performing multi-target assay panels, in particular multiplex vaginal panels (MVP) and methods of use and manufacture. Such cartridges can include a cartridge body having multiple chambers and rotatable valve assembly to facilitate controlled fluid flow, and various reagents and buffers to chemically lyse nucleic acids from multiple targets in MVP. Specialized valve assemblies have been utilized to resist alkaline lysing buffers required to chemically lyse multiple targets, including various protozoa, yeasts and bacteria, including Atopobium spp. (Atopobium vaginae, Atopobium novel species CCUG 55226); Bacterial Vaginosis-Associated Bacterium 2 (BVAB2); and Megasphaera-1. Methods include detecting levels of each respective target in a single sample from a single multi-target assay cartridge and identifying the subject as having bacterial vaginosis, vulvovaginal candidiasis (or the presence of Candida spp.), or trichomoniasis (or presence of Trichomonas vaginalis) based on detected levels of each of: Atopobium spp., BVAB2, Megasphaera-1, Candida spp., and Trichomonas vaginalis.

### CROSS-REFERENCES TO RELATED APPLICATIONS

This application is a Non-Provisional of and claims the benefit of priority of U.S. Provisional Application No. 63/343,969 filed May 19, 2022, the entire contents of which are incorporated herein by reference.

### SEQUENCE LISTING

The contents of the electronic sequence listing (085430-1382316-017910US_ST26.xml; Size: 45,145 bytes; and Date of Creation: Aug. 3, 2023) is herein incorporated by reference in its entirety.

### BACKGROUND OF THE INVENTION

The present invention relates generally to the field of biochemical analysis devices, and in particular to specialized assay cartridges targeted for a multi-target assay panel, in particular a multiplex vaginal panel (MVP).

The analysis of fluids such as clinical or environmental fluids for analytical testing generally involves a series of processing steps, which may include chemical, optical, electrical, mechanical, thermal, or acoustical processing of the fluid samples and subsequent analytical detection of one or more target analytes. Whether incorporated into a bench-top instrument, a disposable cartridge, or a combination of the two, such processing typically involves complex fluidic assemblies and processing algorithms.

Conventional systems for processing fluid samples can employ a number of devices or cartridges, many of which employ a series of chambers each configured for subjecting the fluid sample to a specific processing step. As the fluid sample flows through the system sequentially from chamber to chamber, the fluid sample undergoes the processing steps according to a specific protocol. In some such systems, a sample processing device or cartridge can be operated according to any number of different protocols as required to prepare and test for a given target analyte. These different protocols may require differing buffers, reagent, primers, probes, lysis by chemical and/or mechanical means, as well as various thermal cycling processing.

In recent years, there has been considerable development in the field of biological testing devices that facilitate manipulation of a fluid sample within a sample cartridge to prepare the sample for biological testing by polymerase chain reaction (PCR). One notable development in this field is the GeneXpert sample cartridge by Cepheid. The configuration and operation of these types of cartridges typically involves manipulation of fluid between multiple chambers by a valve assembly, which can be further understood by referring to U.S. Pat. No. 6,374,684 entitled “Fluid Control and Processing System,” and U.S. Pat. No. 8,048,386 entitled “Fluid Processing and Control.” While such sample processing cartridges represent a considerable advancement in the state of the art, there are certain challenges in regard to performance and use of such systems and processes. For example, the precise requirements of different target types (e.g. yeast, bacterial, viral) may require certain processing methods and compounds that may or may not be compatible with a given sample cartridge. Another difficulty is that a particular combination of elements (e.g. buffers/primers/probes/etc.) within a cartridge suited for detection of one target analyte may not be compatible with the combination of elements required for detection of another target analyte. Therefore, some assays may be limited to the differing types of target analytes that can be detected by a single cartridge, thus necessitating obtaining multiple samples and using multiple cartridges each suited for detection of a particular class or type of target analyte. For conditions requiring the detection of multiple targets in an assay panel, these drawbacks associated with conventional cartridge configurations may require multiple samples and cartridges in order to identify a given condition. While various methods and assay panels have been proposed, no single cartridge assay panel has previously been developed that can be performed efficiently from a single sample to reliably identify a suitably broad range of targets needed for a multiplex vaginal panel (MVP).

Particularly, bacterial vaginosis (BV), trichomoniasis and vulvovaginal candidiasis (VVC) are diseases characterized by vaginal discharge. BV is a polymicrobial syndrome in which normal vaginal flora (gram-positive bacteria) are replaced by anaerobic, mostly gram-negative bacteria such as Gardnerella vaginalis, Prevotella species, and Atopobium vaginae. Ultimately, BV is triggered not by just the presence of the potential pathogens but due to an unrestrained increase in their number. As a polymicrobial condition, BV can be diagnosed by assessment of multiple organisms or morphotypes, such as Nugent scoring (assessment and quantification of both gram-positive bacilli and gram-negative curved bacilli) and nucleic acid amplification tests based on detection of multiple organisms. Fredricks et al. (J Clin Microbiol. 2007; 45(10):3270-3276), however, shows detection of G. vaginalis provided high sensitivity but poor specificity of only 29.5%. Other well-established etiologic agent of vaginitis are Trichomonas vaginalis (trichomoniasis) and Candida species (VVC). Vaginitis co-infections are common, clinical symptoms difficult to diagnose, and treatments are different. Therefore, there is clinical value in detecting all three etiologic agents in the same test.

Women presenting with vaginal discharge are often diagnosed at the point of care using a combination of microscopic examination of discharge, and a gross examination of characteristics of discharge (appearance, pH, and odor). Microscopic examination of discharge involves preparation of an unstained wet mount and examination under low power magnification (40×) for clue cells (squamous epithelial cells coated with “sticky” bacteria associated with BV), fungal elements such as hyphae and budding yeast, and motile trichomonads. Amsel criteria constitute some of these characteristics, and are indicators of BV: thin, white to yellow, homogenous discharge; clue cells; pH>4.5; and fishy odor before or after addition of 10% potassium hydroxide. Presence of at least three of these four characteristics represents a positive Amsel test for BV. The wet mount and Amsel criteria are reasonably specific, but rather insensitive tests for BV, VVC, and trichomoniasis. In addition to wet mount microscopy, a Gram stain slide of vaginal secretions can be assessed under high power magnification (1000×) for large gram-positive rod shaped (Lactobacillus sp. representing “good” bacteria) and gram-negative curved rods (representing “bad” bacteria). The smear is scored (Nugent score) based on relative abundance of the two bacterial morphological groups. An abundance of gram-positive bacteria is rated a low Nugent score, while an abundance of gram-negative bacteria is rated a high Nugent score and is indicative of BV. The Gram stain and Nugent score must be performed in a laboratory accredited for high-complexity testing.

Additional tests for BV include Lateral flow immunoassay (OSOM BVBlue, Sekisui Diagnostics) that detects vaginal fluid sialidase. Performance of the OSOM test is comparable to Gram stain. This test is not widely used, despite its CLIA waived status. A DNA probe assay that detects G. vaginalis (a marker for vaginal dysbiosis) has also been developed. While G. vaginalis is present in nearly all cases of BV, it is also frequently found in healthy women. Hence, the high sensitivity (ability of the test to correctly identify those who have the disease) but poor specificity (ability of the test to correctly identify those who do not have the disease) of tests that are based on the detection of G. vaginalis. More recently nucleic acid amplification tests have been developed that detect both “bad” bacteria (anaerobes) and “good” bacteria (lactobacilli) to provide a diagnosis of BV. Software algorithms interpret the results of the different targets and provide a diagnosis. These algorithms provide higher specificity for BV diagnosis than the detection of G. vaginalis alone, but still significantly below 100%.

In addition to wet mount microscopy, trichomoniasis is diagnosed by culture, antigen detection by lateral flow immunoassay, and DNA detection. Culture is performed in special media in clear plastic pouches which are then observed under magnification for characteristic motile organisms. Culture is highly sensitive, and often used as a reference method for diagnosis of trichomoniasis. However, culture can be expensive, time consuming and often inconsistent. A DNA probe assay that detects T vaginalis has also been developed, but its sensitivity is poor in asymptomatic infections and only marginally better in symptomatic persons. Nucleic acid amplification tests that detect T vaginalis provide much higher sensitivity than the DNA probe assay.

There is a need for sample cartridges that overcome various challenges noted above and allow for greater versatility in the number and type of target analytes that can be performed with a single assay cartridge. There is further need for sample cartridges that are specially configured for detection of multiple target analytes required for a given assay panel. There is further need for devices that perform a wide range of sample processing steps in a robust and consistent manner, that accommodate the demands of a comprehensive multi-target assay workflow, and that are compatible with existing technologies to reduce costs and improve patient access.

### BRIEF SUMMARY OF THE INVENTION

The present invention pertains to a multiplex vaginal panel (MVP) assay cartridge and associated methods to aid in assessing vaginal infections in women with a clinical presentation consistent with bacterial vaginosis (BV), vulvovaginal candidiasis, or trichomoniasis, and for the differential diagnosis of these conditions. While this application refers to the term “assay cartridge,” it is appreciated that the term may be used interchangeably with the term “test cartridge” for conducting various types of tests.

In one aspect, the invention pertains to a molecular method for identifying or differentially diagnosing bacterial vaginosis-associated species, vulvovaginal candidiasis-associated species, trichomoniasis-associated species, or a combination thereof in a biological sample obtained from a subject. Such molecular methods can entail: placing the biological sample in a cartridge including a cartridge body having a plurality of chambers in fluidic communication, a reaction vessel configured for amplification of the nucleic acid by thermal cycling, a fluidic path between the plurality of chambers and the reaction vessel, and a filter in the fluidic path; lysing cells in the biological sample with one or more lysis reagents present within at least one of the plurality of chambers and capturing nucleic acid (e.g., DNA) on the filter released therefrom; amplifying the DNA with primers and probes to detect the level of BVAB2, Megasphaera-1, Atopobium spp., Candida spp., and Trichomonas vaginalis in the biological sample, where the primers and probes are present within at least one amplification region of the reaction vessel; and identifying the presence of bacterial vaginosis or bacterial vaginosis-associated species, Candida spp. or candidiasis-associated species, Trichomonas vaginalis or trichomoniasis-associated species, or a combination thereof, or determining that bacterial vaginosis, Candida spp., or Trichomonas vaginalis is not present, in the biological sample based on the detected levels of each of: Atopobium spp., BVAB2, Megasphaera-1, Candida spp. and Trichomonas vaginalis. In some embodiments, Candida spp. refers to antifungal resistant strains.

A positive result for bacterial vaginosis is reported when DNA from the following organism(s) is/are present at an elevated concentration and within their respective Ct ranges: Atopobium vaginae and/or Atopobium novel species; or Atopobium vaginae and/or Atopobium novel species and Megasphaera-1; or Atopobium vaginae and/or Atopobium novel species and BVAB2; or Atopobium vaginae and/or Atopobium novel species and Megasphaera-1 and BVAB2. In some embodiments, bacterial vaginosis is identified if (i) the detected level of Atopobium spp. is at least a first threshold, or (ii) the detected levels of Atopobium spp. is at least a second threshold that is lower than the first threshold and the detected level of BVAB2 or Megasphaera-1 is at least a third threshold, or (iii) the detected levels of Atopobium spp. is at least a fourth threshold that is lower than the second threshold and both BVAB2 and Megasphaera-1 are detected at levels that are at least a fifth threshold. In some embodiments, the threshold can represent the amount of target present in the sample. For example, bacterial vaginosis can be identified if (i) the detected level (first threshold) of Atopobium spp. is at least 320,000 CFU/mL in the absence of BVAB2 and Megaspharea-1, or (ii) the detected levels (second threshold) of Atopobium spp. is at least 2,750 CFU/mL and the detected level (third threshold) of BVAB2 is at least 50 copies/mL and/or of Megasphaera-1 is at least 390 copies/mL.

In some examples, the threshold can represent a cycle threshold (Ct) range/cut-off of the target species. Particularly, the cartridge may utilize two optical channels (Atop gp and Megal-BVAB2 channels) and a BV rules-based algorithm to detect organisms associated with BV and identify a subject having BV (individual organisms may not be reported). For example, the Atop gp channel can include probe(s) specific for detecting Atopobium group (A. vaginae and Atopobium novel species CCUG 55226). The Megal-BVAB2 channel can include probe(s) specific for detecting Megasphaera Type 1 (Megasphaera-1) and Bacterial Vaginosis-Associated Bacterium 2 (BVAB2). In some instances, the test will not report separate results or separate Ct values for Megasphaera Type 1 and BVAB2. A BV positive result can be reported either when both optical channels report a cycle threshold (Ct) within their respective valid Ct ranges, or when only Atopobium group optical channel reports a Ct within its valid Ct range and Megal-BVAB2 Ct is 0 (for the latter scenario, the Atopobium group has an earlier cut-off than that in the presence of Megasphaera-1 and/or BVAB2). For example, bacterial vaginosis can be identified if a Ct range/cut-off for Atopobium spp. in the absence of Megasphaera-1 and BVAB2 is equal to or less than 26 (such as from 8 to 26); or a Ct range/cut-off for Atopobium spp. is equal to or less than 33 (such as from 8 to 33) in the presence of Megasphaera-1 and/or BVAB2, which Ct range/cut-off is equal to or less than 42 (such as from 8 to 42).

In some embodiments, the Candida spp. comprise Candida albicans, Candida dubliniensis, Candida tropicalis and Candida parapsilosis and Candida spp./vulvovaginal candidiasis/vulvovaginal candidiasis-associated species is identified if levels of Candida albicans are at least a first threshold, the levels of Candida dubliniensis are at least a second threshold higher than the first threshold, the levels of Candida tropicalis are at a third threshold between the first and second, and levels of Candida parapsilosis are at a fourth threshold about the same or greater than the second threshold. In some embodiments, the threshold can represent the amount of target present in the sample. For example, Candida spp./vulvovaginal candidiasis/vulvovaginal candidiasis-associated species can be identified if a level of Candida albicans is at least 30 CFU/mL, or a level of Candida dubliniensis is at least 1,316 CFU/mL, or a level of Candida tropicalis is at least 750 CFU/mL, and/or a level of Candida parapsilosis is at least 1,339 CFU/mL. In some examples, the threshold can represent a cycle threshold (Ct) range/cut-off of the target species. Particularly, the cartridge may utilize a single optical channel (Candida group) to detect Candida spp./vulvovaginal candidiasis/vulvovaginal candidiasis-associated species. Individual organisms associated with Candida may not be reported. For example, two sets of oligos can be used to detect Candida spp, one set for the detection of C. albicans, C. tropicalis, C. parapsilosis, C. dubliniensis that targets a segment within the ribosomal protein L19 gene on C. albicans Chromosome 3; the other set for the detection of C. albicans only, and it targets a region in the C. albicans mitochondrial DNA gene, Region Ca-19. A positive detection of Candida spp./vulvovaginal candidiasis/vulvovaginal candidiasis-associated species can be reported either when the optical channel report a cycle threshold (Ct) range/cut-off for C. albicans, C. tropicalis, C. parapsilosis, C. dubliniensis of equal to or less than 40 (such as from 8 to 40); or a Ct range/cut-off for C. albicans of equal to or less than 40 (such as from 8 to 40).

In some embodiments, trichomoniasis/trichomoniasis-associated species/Trichomonas vaginalis is identified if the levels of Trichomonas vaginalis is at least 5 cells/mL. In some examples, the cartridge may utilize a single optical channel to detect trichomoniasis/trichomoniasis-associated species/Trichomonas vaginalis. A positive detection of trichomoniasis/trichomoniasis-associated species/Trichomonas vaginalis can be reported when the optical channel reports a cycle threshold (Ct) range/cut-off for Trichomonas vaginalis of equal to or less than 45 (such as from 13 to 45).

In some embodiments, the method further includes detecting nucleic acid sequences characteristic of antifungal resistant Candida spp. comprising Candida glabrata and Candida krusei in a subject, where the subject is identified as having antifungal resistant Candida spp./antifungal resistant Candida associated species/a second group of Candida spp if level of Candida glabrata is at least 20 CFU/mL and/or Candida krusei at least 656 CFU/mL. In some examples, the cartridge may utilize a single optical channel (Candida glab-krus) to detect antifungal resistant Candida spp. (Candida glabrata and Candida krusei). One oligo set can be specific for the detection of C. glabrata and the oligos target tandem repeat regions on chromosome L; and another oligo set can be specific for C. krusei, targeting the coding region of a hypothetical protein, possibly a helicase.

The biological sample can be a vaginal swab, a vaginal mucus sample, a vaginal tissue sample, a vaginal cell sample, or a biological sample collected from the urethra, penis, anus, throat, or cervix. It is appreciated that in some embodiments, the cartridge can be configured to detect and identify one of bacterial vaginosis, vulvovaginal candidiasis, or trichomoniasis, or any combination thereof, in accordance with the thresholds and levels described above. In other embodiments, the cartridge can be configured to detect and identify bacterial vaginosis, Candida spp. (individual organisms associated with Candida may not be reported), or Trichomonas vaginalis, or any combination thereof, in accordance with the thresholds and levels described above.

In some embodiments, the cartridge provides lysis of viral, bacterial, parasitic protozoan, fungal, and epithelial cells. Advantageously, detection can be effected without transporting the sample from the site where the sample is collected (e.g., a POC diagnosis is preferably a hospital, an urgent care center, an emergency room, a physician's office, a health clinic, or a home). The method can include a Clinical Laboratory Improvement Amendments (CLIA)-waived test. The cartridge can be a Clinical Laboratory Improvement Amendments (CLIA)-compliant cartridge, is operated in compliance with CLIA, is operated by a CLIA-compliant laboratory, or is operated in a CLIA-compliant location. The cartridge can be a Clinical Laboratory Improvement Amendments (CLIA)-certified device, is operated by a CLIA-certified laboratory, is operated in a CLIA-certified location, is operated under the oversight of a CLIA-compliant laboratory, or is operated under the oversight of a CLIA-certified laboratory.

In some embodiments, the cartridge and methods herein allow the presence or absence of bacterial vaginosis, vulvovaginal candidiasis/Candida spp., trichomoniasis/Trichomonas vaginalis, or a combination thereof to be detected within the biological sample within 90 minutes, 75 minutes, 60 minutes, 45 minutes, 30 minutes or 15 minutes of collecting the sample from the subject. The method can provide results for a positive or negative bacterial vaginosis, vulvovaginal candidiasis/Candida spp., antifungal resistant candidiasis/Candida glabrata/Candida krusei, or trichomoniasis/Trichomonas vaginalis diagnosis. In some instances, the method does not provide for an indeterminate BV diagnosis. Typically, the method has a false negative rate of less than 20%, less than 10%, or less than 5%. In some examples, the method can provide identification of bacterial vaginosis, Candida spp., or Trichomonas vaginalis in the biological sample with a sensitivity of at least 90% and a specificity of at least 90%, preferably with a sensitivity of at least 95% and a specificity of at least 95%, based on the detected levels of each of: Atopobium spp., BVAB2, Megasphaera-1, Candida spp., and Trichomonas vaginalis.

In some embodiments, the sample preparation method can include lysis reagents that are a chaotropic agent, a chelating agent, a buffer, an alkaline agent, or a detergent. The chaotropic agent can be selected from a guanidinium compound such as guanidinium thiocyanate or guanidinium hydrochloride, an alkali perchlorate such as lithium perchlorate, an alkali iodide, magnesium chloride, urea, thiourea, a formamide, or a combination thereof. The lysis reagents can include a guanidinium compound, sodium hydroxide, EDTA, a buffer, and a detergent. When present, the sodium hydroxide may be isolated (away from guanidinium compound, EDTA, a buffer, and/or a detergent) in a single chamber prior to lysing the biological sample. In some examples, the sodium hydroxide can be mixed with the lysis reagent once the test is started. In some embodiments, the cartridge can include magnetic beads. In some embodiments the cartridge includes a filter that can be configured to bind the nucleic acid to be analyzed. The filter can include glass fibers and optionally a polymeric binder, or the glass fibers are optionally modified with a DNA binding ligand such as an alkylamine, a cycloalkylamine, an alkyloxy amine, a polyamine moiety, an arylamine, an intercalating agent, a DNA groove binder, a peptide, an amino acid, a protein, or a combination thereof. The filter can be a 500 microns to 2000 microns thick glass fiber disk having a pore size of 0.2 microns to 1 micron. In some embodiments, the filter is configured to bind unwanted material and allowing the nucleic acid to pass through. In some embodiments, the filter is configured to capture and isolate the nucleic acid. In some embodiments, the filter can be derived from any suitable material, including but not limited to glass, silica, titanium oxide, aluminum oxide, iron oxide, ethylenic backbone polymers, polypropylene, polyethylene, polystyrene, ceramic, cellulose, nitrocellulose, magnetic silica particles, and divinylbenzene. In some embodiments, the filter comprises a material selected from polystyrene, glass, ceramic, polypropylene, polyethylene, silica, mica, titanium dioxide, polycarbonate, latex, PMMA, zeolite, polyethersulfone, carboxymethylcellulose, cellulose, and combinations thereof. Examples of solid support includes a magnetic bead, a glass bead, a polystyrene bead, cellulose filter, a polystyrene filter, a polycarbonate filter, a polyethersulfone filter, polytetrafluoroethylene filter, polyvinylpyrrolidone filter, or a glass filter. The filter may further comprise a polymeric binder for binding the particles or fibers in the filter. Exemplary polymeric binders include an acrylic polymer.

The methods and cartridge can utilize any of a binding reagent, wash reagent, eluting reagent, or a combination thereof. The eluting reagent can include ammonia or an alkali metal hydroxide. In some embodiments, the eluting reagent has a pH above about 8, above about 9, above about 10, or above about 11. The eluting reagent can include a polyanion, preferably a carrageenan, a carrier nucleic acid, or i-carrageenan and KOH.

In some embodiments, the methods herein utilize various primers and probes. Primers and probes for amplifying and detecting Atopobium spp. are capable of hybridizing to the 16S rRNA gene of Atopobium vaginae. Particularly, the candidate region towards the 5′ end of the 16S rRNA gene (GenBank accession number HM007563.1, coordinates 36 to 151) was identified based on conservation and the pattern of oligo Tms, allowing detection of Atopobium novel species along with Atopobium vaginae in the Atopobium group. The primers and probes for amplifying and detecting BVAB2 are capable of hybridizing to the 16S rRNA gene of BVAB2 (GenBank accession AY724740.1). Particularly, a region showing a number of clear differences between BVAB2 and BVAB1/BVAB3 and was selected as the target sequence for BVAB2. The primers and probes for amplifying and detecting Megasphaera-1 are capable of hybridizing to the 16S rRNA gene of Megasphaera-1 (representative sequences for Megasphaera Type 1 and Type 2 include GenBank accession numbers EF120358 and EF120359, respectively). Particularly, a central “core” region (˜150 nucleotide) showing a number of clear differences between Megasphaera Type 1 and Type 2 was extracted as the target region for the detection of Megasphaera-1. The primers and probes for amplifying and detecting Candida spp. include targets that are capable of hybridizing to the Region Ca-19 (Mitochondrial DNA) gene of Candida albicans, tandem repeat regions on chromosome L gene of Candida glabrata, and coding region of a hypothetical protein of Candida krusei. A “universal” Candida oligo set was not identified, as they are located on different branches of the phylogenetic tree and would cross-react with other fungal genomes such as Saccharomycetes. A segment that is conserved among C. albicans, C. dubliniensis, and C. tropicalis and yet is distinct from others within the coding sequence of ribosomal protein L19 was identified as a target to reduce the number of oligos as well as reduce cross-reactivity with other fungal genomes. Ca19-mtDNA “mitochondrial DNA, Region Ca-19” was selected as the target sequence for the detection of C. albicans since it was identified as being highly specific to C. albicans and did not show significant similarity to other Candida species, including C. dubliniensis. The target region for Candida glabrata on Chromosome L was found in 10 identical tandem repeats in a specific genome location, making it a good target. Pichia kudriavzevii is the current preferred name for C. krusei. The amplicon for C. krusei appears to lie in the coding region of a hypothetical protein, possibly a helicase. The primers and probes for amplifying and detecting the presence of Trichomonas vaginalis are capable of hybridizing to the 40S ribosomal protein S23 gene of Trichomonas vaginalis (Genbank accession number XP001317004).

In some embodiments, the primers and probes for amplifying and detecting the presence of Atopobium vaginae comprise a sequence that is identical or complementary to at least 15 contiguous nucleotides of one or more of SEQ ID NOs: 1, 2, and 3; the primers and probes for amplifying and detecting the presence of BVAB2 comprise a sequence that is identical or complementary to at least 15 contiguous nucleotides of one or more of SEQ ID NOs: 4, 5, and 6; the primers and probes for amplifying and detecting the presence of Megasphaera-1 comprise a sequence that is identical or complementary to at least 15 contiguous nucleotides of one or more of SEQ ID NOs: 7, 8, and 9; the primers and probes for amplifying and detecting the presence of Candida albicans comprise a sequence that is identical or complementary to at least 15 contiguous nucleotides of one or more of SEQ ID NOs: 10, 11, 12, 13, 14, 15, and 16; the primers and probes for amplifying and detecting the presence of Candida glabrata comprise a sequence that is identical or complementary to at least 15 contiguous nucleotides of one or more of SEQ ID NOs: 17, 18, and 19; the primers and probes for amplifying and detecting the presence of Candida krusei comprise a sequence that is identical or complementary to at least 15 contiguous nucleotides of one or more of SEQ ID NOs: 20, 21, and 22; and the primers and probes for amplifying and detecting the presence of Trichomonas vaginalis comprise a sequence that is identical or complementary to at least 15 contiguous nucleotides of one or more of SEQ ID NOs: 23, 24, and 25.

In some embodiments, the method utilizes primers and/or probes comprising a detectable label. The primers and/or probes can include a fluorescent dye and a quencher molecule. The reaction vessel can utilize a primer pair for detecting an exogenous control and/or an endogenous control, where the exogenous control is a sample processing control, and where the endogenous control is a sample adequacy control. In some embodiments, the method further utilizes primers and/or probes that detect nucleic acid sequences characteristic of Lactobacillus bacteria and/or Gardnerella vaginalis. In some embodiments, the methods do not detect nucleic acid sequences characteristic of any Lactobacillus bacteria or Gardnerella vaginalis, although these targets can optionally be included in other embodiments. The amplification can be by a real-time PCR multiplex assay. The methods can further include administering a treatment regimen to the subject based on the assessed diagnosis or organisms detected. Particularly, the methods can further include administering an antibiotic and/or antifungal treatment (such as metronidazole, clindamycin, clotrimazole, or miconazole).

In another aspect, the invention pertains to a cartridge configured for performing a multiplex vaginal panel (MVP). In some embodiments, the MVP is designed for identifying bacterial vaginosis, vulvovaginal candidiasis/Candida spp., trichomoniasis/Trichomonas vaginalis, or a combination thereof in a subject. The cartridge can include a cartridge body having a plurality of chambers defined therein, where the plurality of chambers are in fluidic communication through a fluidic path of the cartridge, a reaction vessel configured for amplification of the nucleic acid by thermal cycling and having a reaction chamber therein, where the reaction vessel is attached to the cartridge body and fluidically coupled to the fluidic path of the cartridge; and a filter disposed in the fluidic path between the plurality of chambers and the reaction vessel. In some embodiments, the plurality of chambers and/or the reaction chamber independently comprise lysis reagents for releasing nucleic acid from a sample, and primers and probes for amplifying and detection of DNA sequences characteristic of BVAB2, Megasphaera-1, Atopobium spp., Candida spp., and Trichomonas vaginalis.

In some embodiments, the assay cartridge includes: a cartridge body having a plurality of chambers therein, the plurality of chambers including: a sample chamber having at least a fluid outlet in fluid communication with another chamber of the plurality; a lysis chamber in fluidic communication with the sample chamber, the lysis chamber including lysis reagents for releasing nucleic acid, optionally where the sample chamber and lysis chamber are the same; a reaction vessel fluidically coupled to the plurality of chambers of the cartridge body and configured for amplification of nucleic acid by thermal cycling; a filter disposed in the fluidic path between the lysis chamber and the reaction vessel; and a plurality of primers and/or probes disposed in one or more amplification regions of the reaction vessel for detection of DNA sequences characteristics of BVAB2, Megasphaera-1, Atopobium spp., Candida spp., and Trichomonas vaginalis. In some embodiments, the cartridge body further includes a base portion that allows the cartridge to stand in an upright orientation without further support and can also include a top lid having a snap fit end and a hinged end opposite the snap fit. In some embodiments, the top lid includes an upper portion and a lower portion connected via the hinged end, the upper portion being moveable from an open position to a closed position and the lower portion has at least one major port providing access to at least one of the plurality of chambers. In some embodiments, the cartridge body further includes a valve assembly configured to rotate and having at least two ports to fluidically connect the chambers. Preferably, the valve assembly is resistant to alkali corrosion. In some embodiments, the valve assembly can be formed from a polycarbonate material annealed, for example, by heating to a temperature of at least 100° C. for at least 1 hour and controlled cooling.

In some embodiments, the cartridge body further comprises one or more burstable seals and/or one or more heat labile membrane, to fluidically connect the chambers and/or to cover the cartridge body. The cartridge body can be configured to allow for controlled pumping of components received in each chamber over the plurality of chambers. In some embodiments, the cartridge includes at least one chamber that is deformable and configured to hold a fluid therein when in an undeformed state and to collapse upon application of an external compression force to expel at least a portion the fluid from the at least one chamber.

Cell lysis is performed to break or rupture the cells in the sample to provide a gateway through which the cell's components are extracted. Cell lysis can be performed either chemically, mechanically, or electromechanically, or combinations thereof. In some embodiments, the lysis chamber can comprise an ultrasonic, piezoelectric, magnetostrictive, or electrostatic transducer within the cartridge or interface of the module. In some aspects, the lysis chamber includes an ultrasonic transducer that can output ultrasonic waves in a frequency range to lyse cells obtained from the biological sample within the lysis chamber (such as from approximately 2800 kHz to approximately 3200 kHz). The specific frequency for lysing the cells can be predetermined, depending on the specific cells. In other aspects, the cells are lysed by lysis reagents. The lysis reagents can include a chaotropic agent, a chelating agent, a buffer, and a detergent. The chaotropic agent can be selected from guanidinium thiocyanate, guanidinium hydrochloride, alkali perchlorate, alkali iodide, urea, formamide, or combinations thereof. The lysis reagents can include a guanidinium compound, sodium hydroxide, EDTA, a buffer, or a detergent. The lysis reagents can further include magnetic beads. For example, the filter is configured to bind the nucleic acid to be analyzed and can include glass fibers and optionally magnetic beads and/or a polymeric binder (such as an acrylic polymer). The glass fibers can be optionally modified with a DNA binding ligand such as an alkylamine, a cycloalkylamine, an alkyloxy amine, a polyamine moiety, an arylamine, an intercalating agent, a DNA groove binder, a peptide, an amino acid, a protein, or a combination thereof. In other instances, magnetic beads can be added to the liquid reagent to aid with nucleic acid extraction. The filter can be a 500 microns to 2000 microns thick glass fiber disk having a pore size of 0.2 microns to 1 micron. Preferably, the filter is configured to bind unwanted material and allowing the nucleic acid to pass through. The filter can be formed as a polymeric disk or glass fiber disk or optionally as glass beads. The cartridge can further include a binding reagent, wash reagent, eluting reagent, or a combination thereof. The eluting agent comprises ammonia or an alkali metal hydroxide. Typically, eluting agent has a pH above about 8, above about 8.5, above about 9, above about 10, or above about 11. The eluting agent can include a polyanion, preferably a carrageenan, a carrier nucleic acid, or i-carrageenan and KOH. In further aspects, the cells can be lysed by a combination of mechanical and chemical lysis. For example, the cells can be lysed using a frequency range of about 40 kHz or less (such as about 30 kHz to about 40 kHz) in combination with a lysis reagent as described herein.

In some embodiments, the cartridge includes a reaction tube or vessel extending from the cartridge body. Typically, the reaction vessel is positioned in a vertical orientation, and includes an inlet and an outlet, where the outlet is positioned above the inlet when the tube is in a vertical orientation. The reaction vessel can include an optical window for monitoring the progress of the reaction. The reaction vessel can further include at least one amplification region for analyzing a nucleic acid, or can include at least two (such as three or four) independent amplification regions. Typically, the reaction vessel has two major faces separated by a plurality of minor walls. The reaction vessel is configured such that thermal energy is transmitted into the reaction vessel through the major faces. In some instances, the reaction vessel has a single face or a single major face through which thermal energy is transmitted into the reaction vessel. In other embodiments, the reaction vessel is configured such that a steady state temperature gradient can be formed in a manner conducive for amplification. For example, the reaction vessel can be configured to perform amplification by creating a convection current within a reaction chamber by generating a fixed high temperature and fixed low temperature at different locations within the reaction chamber. In other embodiments, the reaction vessel can be configured to allow for amplification by movement of the fluid using, for example, a fluid pump, from one reaction vessel to another wherein each vessel is heated at a different temperature. In further embodiments, the reaction vessel together with the choice of primers and probes can be configured to allow for amplification to occur under isothermal conditions, without thermocycling. In some embodiments, the reaction vessel is optically monitored through the minor or major walls. In some embodiments, each component of the cartridge is stable from 2-30° C. for at least 5 months, preferably at least 9 months, most preferably, at least 12 months. The cartridge can be a single-use disposable cartridge. Typically, the cartridge is configured to perform amplification and detection by a real-time PCR multiplex assay.

In some embodiments, the cartridge can include at least one of the plurality of chambers that include lyophilized reagents. The lyophilized reagents can be in the form of one or more beads. The lyophilized reagents are selected from reagents, primers and probes, salts, dNTPs, enzymes such as a thermostable polymerase or a reverse transcriptase, or a combination thereof. The amplification region can also include lyophilized primers and probes. In certain embodiments, lyophilized lysis reagents such as enzymes, buffers, chaotropic agents, salts, or a combination thereof can be in the form of one or more beads and present inside one or more of the chambers. In certain embodiments, lyophilized binding and/or wash reagents such as buffers, salts, polyalkylene glycol, ethanol, or a combination thereof can be in the form of one or more beads and present inside one or more of the chambers. In certain embodiments, reagents including primers and probes can be spotted and lyophilized inside the reaction chamber(s). The reagents and components in the plurality of chambers of the cartridge and/or the reaction vessel can be in solution.

In some embodiments, the cartridge includes various primers and probes, including any of the following or any combination thereof: primers and probes for amplifying and detecting Atopobium spp. are capable of hybridizing to the 16S rRNA gene of Atopobium vaginae; primers and probes for amplifying and detecting BVAB2 are capable of hybridizing to the 16S rRNA gene of BVAB2; primers and probes for amplifying and detecting Megasphaera-1 are capable of hybridizing to the 16S rRNA gene of Megasphaera-1; primers and probes for amplifying and detecting Candida spp. are capable of hybridizing to the Region Ca-19 (Mitochondrial DNA) gene of Candida albicans, tandem repeat regions on chromosome L gene of Candida glabrata, and coding region of a hypothetical protein of Candida krusei; and primers and probes for amplifying and detecting the presence of Trichomonas vaginalis are capable of hybridizing to the 40S ribosomal protein S23 gene of Trichomonas vaginalis.

In some embodiments, the primers and probes include a detectable label. The primers and/or probes can comprise a fluorescent dye and a quencher molecule. The reaction vessel further includes a primer pair for detecting an exogenous control and/or an endogenous control, where the exogenous control is a sample processing control, where the endogenous control is a sample adequacy control. Optionally, the cartridge can include primers and probes to detect nucleic acid sequences characteristic of antifungal resistant Candida spp. In some embodiments, the primers and/or probes further detect nucleic acid sequences characteristic of any Lactobacillus bacteria or Gardnerella vaginalis. In alternate embodiments, the primers and/or probes do not detect nucleic acid sequences characteristic of any Lactobacillus bacteria or Gardnerella vaginalis.

In yet another aspect, the invention pertains to a system for performing a multiplex vaginal panel (MVP) for identifying bacterial vaginosis, vulvovaginal candidiasis/Candida spp., trichomoniasis/Trichomonas vaginalis, or a combination thereof in a subject. In some embodiments, the system includes a module having a receiving bay for receiving the multi-target assay panel cartridge, where the module includes one or more mechansims within the receiving bay for manipulating a fluid sample within the cartridge, and an instrument that interfaces with the reaction vessel; and a memory having programmable instructions recorded thereon, that are specially configured to operate the module according to an MVP assay panel protocol to determine DNA sequence characteristics of BVAB2, Megasphaera-1, Atopobium spp., Candida spp., and Trichomonas vaginalis. The programmable instructions can be configured to perform any of the methods described herein. The system can further include: a scanner or reader configured to read an identifier on the cartridge; where the instructions are configured to determine an applicable protocol based on reading or scanning of the identifier. The system operates the module according to the MVP protocol in response to determining the cartridge is configured for the MVP assay based on an input from the scanner or reader. The system can further include an enclosure that at least partly encloses a plurality of modules that are substantially similar or identical and configured to concurrently perform assays on cartridges received therein. In some embodiments, the modules can have differing configurations (e.g. in regard to temperature cycling capabilities, detection, light sources).

### BRIEF DESCRIPTION OF THE DRAWINGS

FIGS. 1A-1C show an overview of a sample cartridge with a valve assembly configured for performing differing sample processes, including chemical lysing of targets, which is configured for PCR and optional integrated nucleic acid analysis of the target assay panel in accordance with some embodiments of the invention. FIG. 1A shows the sample cartridge body with reaction vessel, FIG. 1B shows an exploded view of the sample cartridge, and FIG. 1C shows components of the valve assembly, in accordance with some embodiments.

FIGS. 2A-2B illustrate details as to the targets being detected for the subject assay panel and an associated assay workflow, in accordance with some embodiments.

FIG. 3 illustrates various valve assemblies A, B, C, D each suited for one or more types of target lysing, any of which may be used in a respective sample cartridge.

FIGS. 4A-4C show illustrative, but non-limiting embodiments of the modules, and systems (e.g., processing units) for the PCR detection and/or quantification of polypeptide(s) and optional integrated nucleic acid analysis for the targeted assay panel. FIG. 4A illustrates a module configured to receive and interact with the valve assembly of the cartridge to operate the cartridge to facilitate sample preparation and analysis. FIG. 4B illustrates a processing unit (e.g. analytical testing unit) of the module that interacts with the fluid sample in the reaction vessel to facilitate sample processing and analytical testing (e.g. PCR and, optionally, nucleic acid analysis) for the targeted assay panel. FIG. 4C illustrates an analytical system having multiple such modules within an enclosure so as to receive multiple sample cartridges therein for testing of the targeted assay panel and/or various other targets or panels.

FIG. 5 shows a non-limiting workflow for PCR and optional nucleic acid analysis (e.g., nucleic acid amplification) of the targeted assay. In some embodiments, PCR and nucleic acid analysis when performed, are both performed on the same sample. Thus, a single sample can be introduced into one sample chamber. In other embodiments the sample may be processed differently for PCR and nucleic acid analysis for the target assay panel.

FIG. 6 depicts additional assay workflows, in accordance with some embodiments.

FIG. 7 illustrates an exploded view of the sample cartridge illustrating its major components, including the lid, multi-chamber body, reaction vessel, valve assembly and base, in accordance with some embodiments. The chambers may be used to perform various processes (e.g. extract, purify, deamidate, desulfonate, MSP) on the sample.

FIGS. 8A-B show specialized components of the cartridge configured for the targeted assay panel, in accordance with some embodiments. The specialized component is a valve assembly having a syringe tube through which the sample is injected and multiple ports on a valve body that facilitate transport of fluid sample between the respective processing chamber upon rotation of the valve assembly.

FIGS. 9-10 show exemplary methods of identifying bacterial vaginosis, vulvovaginal candidiasis/Candida spp., trichomoniasis/Trichomonas vaginalis of a subject by use of the assay panels described herein, in accordance with some embodiments.

### DETAILED DESCRIPTION

The present invention relates generally to a system, device and methods in the field of biochemical analysis for a targeted assay panel, including specialized assay cartridges configured for the targeted assay panel, in particular a multiplex vaginal panel (MVP). The invention further pertains to specialized components and methods of fabrication and assembly of the specialized assay cartridges.

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides. The term nucleic acid includes any form of DNA or RNA, including, for example, genomic DNA; complementary DNA (cDNA), which is a DNA representation of mRNA, usually obtained by reverse transcription of messenger RNA (mRNA) or viral RNA or by amplification; DNA molecules produced synthetically or by amplification; mRNA; and non-coding RNA. The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands). The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5′-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “gene” encompasses coding sequences, introns, and any associated control sequences that participate in the expression of the coding sequences.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Selective hybridization” or “selective annealing” refers to the binding of a nucleic acid to a target nucleic acid in the absence of substantial binding to other nucleic acids present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to “anneal to” or “hybridize to” another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence. This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable moiety to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

The term “target” is used herein with reference to “target nucleic acids,” as well as “target organisms.” The former refers to nucleic acids to be detected, and the latter refers to organisms to be detected. The term, “target nucleic acid” is generally used herein to refer to a segment of nucleic acid that is defined by a primer pair and that gives rise to an amplicon produced in an amplification reaction; the term “amplification target” is also used herein to refer to this type of target nucleic acid. Primers and probes are also said to “target” nucleic acid sequences, and so these sequences can also be understood as “target nucleic acids.” Additionally, primers and probes are said to “target” or “be specific for” genes. In this usage, the primers and probes can be used to detect the presence of a particular gene by specifically hybridizing to a portion of the gene that indicates its presence. The meaning of “target” and “target nucleic acids” will be clear to one of skill in the art from the context in which the term is employed. In some embodiments, multiple target nucleic acids can be detected to detect a single target organism. In some embodiments, a single target nucleic acid can be detected to detect a single target organism. In some embodiments, an assay can employ multiple target nucleic acids for one or more target organisms and single target nucleic acids for one or more different target organisms.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February;4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February;13(2):294-307, and Landegren et al., Science 241:1077-80 (1988), Demidov, V., Expert Rev Mol Diagn. 2002 November;2(6):542-8, Cook et al., J Microbiol Methods. 2003 May; 53(2):165-74, Schweitzer et al., Curr Opin Biotechnol. 2001 February;12(1):21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

A multiplex amplification reaction is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation and produces a detectable signal (e.g., a fluorescent signal).

The term “quencher,” as used herein generally refers to any organic or inorganic molecule that reduces the level of a detectable signal.

As used herein, the term “detecting” refers to “determining the presence of” an item, such as a nucleic acid sequence, e.g., one that is indicative of the presence of an Atopobium spp. Detection can include the determination of the presence of an Atopobium spp, without definitive identification of that Atopobium spp; the determination of the presence of a particular, known Atopobium spp; or determination of the presence of a novel (not previously described) Atopobium spp.

The term “level”, as used herein, refers to an approximate threshold numerical quantity or amount of a target that may be present in a sample. The level can be designated or set at whatever level is necessary for a particular assay. In some examples, the level of a particular target is designated as a Ct value. The term “Ct” as used herein refers to threshold cycle, the cycle number in quantitative polymerase chain reaction (qPCR) at which the fluorescence-generated within a reaction well exceeds the defined threshold, i.e. the point during the reaction at which a sufficient number of amplicons have accumulated to meet the defined threshold. In more specific examples, the level of a target can be defined as the limit of detection (LOD) or near cut-off concentration. The term “LOD” is defined as the lowest concentration of organism sample that can be reproducibly distinguished from negative samples with 95% confidence. The near cut-off concentration for example, of the BV organisms is defined as the lowest concentrations of Atopobium vaginae and Megasphaera-1, or A. vaginae and BVAB2, or A. vaginae and Megasphaera-1 and BVAB2, or A. vaginae in the absence of Megasphaera-1 and BVAB2 that result in BV POSITIVE test results and can be reproducibly distinguished from negative samples with a 95% confidence level.

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA for a human sample. Nonlimiting exemplary endogenous controls include ABL mRNA, GUSB mRNA, GAPDH mRNA, TUBB mRNA, and UPKla mRNA. In some embodiments, an endogenous control, such as an SAC, is selected that can be detected in the same manner as the target nucleic acid (e.g., RNA) is detected and, in some embodiments, simultaneously with the target nucleic acid (e.g., RNA).

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target nucleic acid (e.g., RNA). In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target nucleic acid (e.g., RNA) is detected and, in some embodiments, simultaneously with the target nucleic acid (e.g., RNA). In some embodiments, the exogenous control is an RNA. In some such embodiments, the exogenous control is an Armored RNA®, which comprises RNA packaged in a bacteriophage protective coat. See, e.g., WalkerPeach et al, Clin. Chem. 45: 12: 2079-2085 (1999).

### I. System Overview

In one aspect, the invention pertains to a sample cartridge that utilizes a valve body platform that allows for the detection of enveloped and free nucleic acid targets. In some embodiments, the valve body includes a sample processing region or lysing chamber that provides for either or both mechanical and chemical lysis. This allows a single cartridge to provide lysing for a multitude of differing types of targets, thus, can be considered an “assay panel cartridge.” In some embodiments, the sample cartridge can perform processing and detection of both bacterial targets requiring mechanical lysing and viral targets suited for chemical lysing.

The sample cartridge device can be any device configured to perform one or more process steps relating to preparation and/or analysis of a biological fluid sample according to any of the methods described herein. In some embodiments, the sample cartridge device is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the sample cartridge. In some embodiments, the reaction vessel extends from the body of the cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

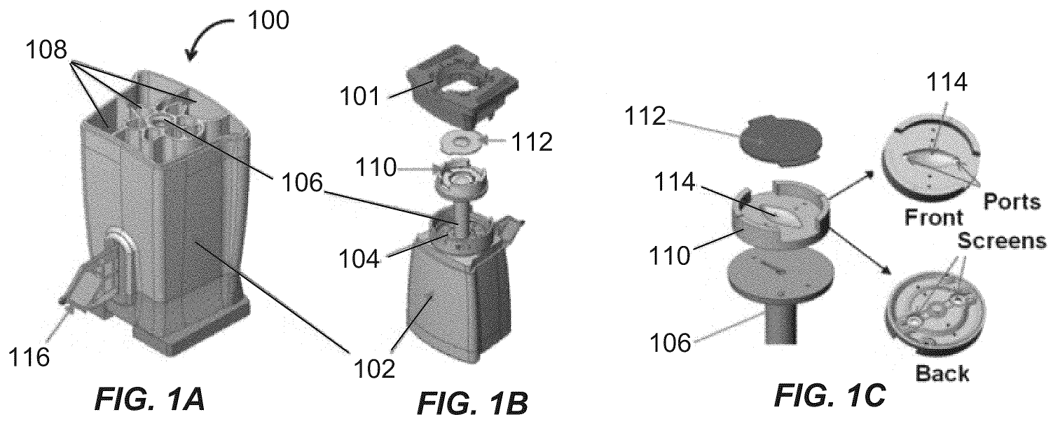

A sample cartridge suitable for use with the invention, includes one or more transfer ports through which the prepared fluid sample can be transported into an attached reaction vessel for analysis. FIG. 1A illustrates an exemplary assay panel cartridge 100 suitable for sample preparation and analytics testing by PCR when received in an instrument module in accordance with some embodiments. The sample cartridge is attached with a reaction vessel 116 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. The instrument module facilitates the processing steps needed to perform sample preparation and the prepared sample is transported through one of a pair of transfer ports into fluid conduit of the reaction vessel 116 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacteria, a virus, a pathogen, a toxin, or other target analyte. The optical detection means can include 4 or more optical channels, preferably 6 or more optical channels, more preferably 10 or more optical channels. It is appreciated that such a reaction vessel could include various differing chambers, conduits, or micro-well arrays for use in detecting the target analyte. The sample cartridge can be provided with means to perform preparation of the biological fluid sample before transport into the reaction vessel. Any chemical reagent required for viral or cell lysis, or means for binding or detecting an analyte of interest (e.g. reagent beads) can be contained within one or more chambers of the sample cartridge, and as such can be used for sample preparation.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/217,672 entitled “Universal Assay Cartridge and Methods of Use” filed Jul. 1, 2021; U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016; the entire contents of which are incorporated herein by reference in their entirety for all purposes. The above noted patents are included in the attached appendix.

Various aspects of the sample cartridge 100 can be further understood by referring to U.S. Pat. No. 6,374,684 “the '684 patent”), which described certain aspects of a sample cartridge in greater detail. Such sample cartridges can include a fluid control mechanism, such as a rotary fluid control valve assembly, that is fluidically connected to the chambers of the sample cartridge. The term “chamber” can be used interchangeably with the terms “well”, “tube”, and the like. Rotation of the rotary fluid control valve permits fluidic communication between chambers and the valve so as to control flow of a biological fluid sample deposited in the cartridge into different chambers in which various reagents can be provided according to a particular protocol as needed to prepare the biological fluid sample for analysis. To operate the rotary valve, the cartridge processing module comprises a motor such as a stepper motor that is typically coupled to a drive train that engages with a feature of the valve in the sample cartridge to control movement of the valve in coordination with movement of the syringe, thereby resulting movement of the fluid sample according to the desired sample preparation protocol. The fluid metering and distribution function of the rotary valve according to a particular sample preparation protocol is demonstrated in the '684 patent.

### II. Example Assay Cartridge and Valve Assemblies

### A. Overview

As shown in FIG. 1A, the assay panel cartridge 100 comprises a cartridge body 102 containing a plurality of chambers 108 for reagents or buffers and sample processing. The chambers are disposed around a central syringe barrel 106 that is in fluid communication with a valve body 110 (see FIGS. 1B and 1C) and that is sealed with a gasket 104. The valve body 110 can include a cap 112 and the entire cartridge body can be supported on a cartridge base 101. The valve body typically contains one or more channels or cavities (chamber(s) 114) that can contain a filter as described herein that can function to bind and elute a nucleic acid. In some embodiments the cartridge further comprises one or more temperature controlled channels or chambers that can, in certain embodiments, function as thermocycling chambers. A “plunger” not shown can be operated to draw fluid into the syringe barrel 106 and rotation of the valve body 110 provides selective fluid communication between the various reagent chambers 108 and channels, reaction chamber(s), mixing chambers, and optionally, any temperature controlled regions. Thus, the various reagent chambers 108, reaction chambers, filter material(s), and temperature controlled chambers or channels are selectively in fluid communication by rotation of the plunger and reagent movement (e.g., chamber loading or unloading) is operated by the “syringe” action of the plunger within the valve assembly. In other embodiments, the various reagent chambers, reaction chambers, filter material, and temperature-controlled chambers or channels are selectively in fluid communication by linear progression (e.g., by forced movement) of the reagents and sample from one chamber to the next.

### B. Multiplex Vaginal Panel (MVP) Assays

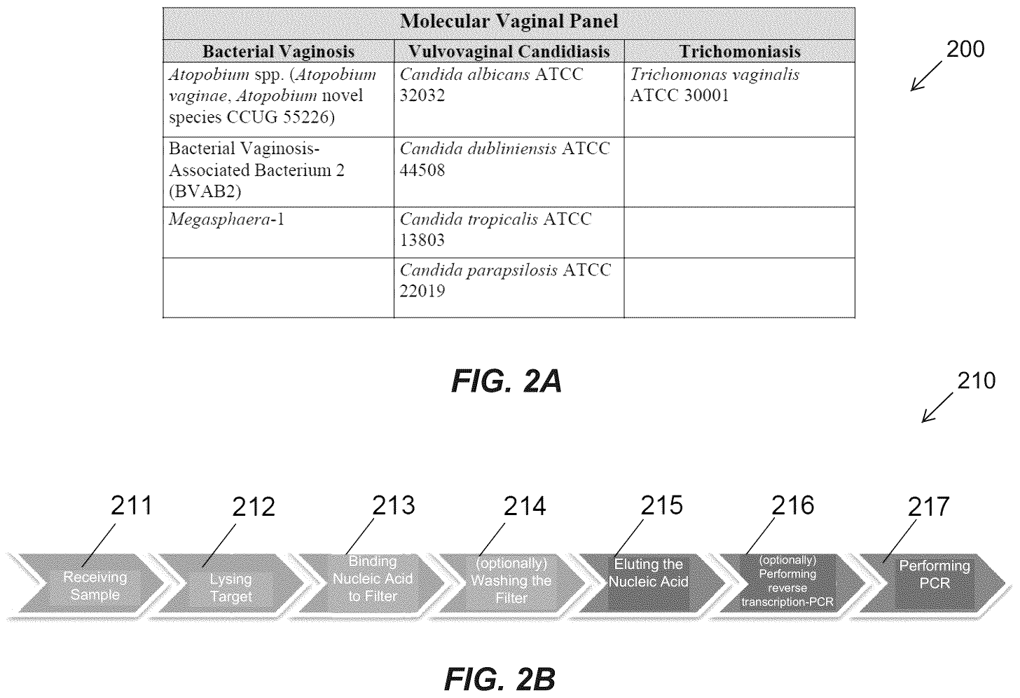

In one aspect, the assay cartridge is configured to support multi-target assay panels developed for use with the sample cartridge. In the embodiments described herein, the assay panel cartridge is an MVP assay cartridge (“MVP cartridge”) configured to perform an MVP assay panel that identifies multiple targets of interest, as detailed in FIG. 2A. In some embodiments, the MVP cartridge is configured to operate on the GeneXpert® system, although it is appreciated these concepts can be applied to any assay cartridge or assay device.

In an exemplary embodiment, the MVP cartridge is an automated qualitative in vitro diagnostic test for the detection of DNA targets from anaerobic bacteria associated with bacterial vaginosis (BV), Candida species associated with vulvovaginal candidiasis, and Trichomonas vaginalis associated with Trichomoniasis. The MVP cartridge may be further configured for the detection of DNA targets from Candida species associated with antifungal resistance. The MVP cartridge test can use clinician-collected or self-collected vaginal swabs collected in a clinical setting from patients who are symptomatic for vaginitis/vaginosis. The MVP cartridge test utilizes real-time polymerase chain reaction (PCR) for the amplification of specific DNA targets and utilizes fluorogenic target-specific hybridization probes to detect and differentiate DNA from organisms associated with vaginitis/vaginosis. It is appreciated that the terms “MVP cartridge test” and “MVP test” are used interchangeably throughout.

1. Summary of MVP

Organisms associated with bacterial vaginosis (detected organisms may not be reported individually) include: Atopobium spp. (Atopobium vaginae, Atopobium novel species CCUG 55226); Bacterial Vaginosis-Associated Bacterium 2 (BVAB2); and Megasphaera-1. Organisms associated with vulvovaginal candidiasis (detected organisms may not be reported individually) include: Candida spp. (C. albicans, C. tropicalis, C. parapsilosis, C. dubliniensis). Organisms associated with Candida antifungal resistance (detected organisms may not be reported individually) include: Candida glabrata and Candida krusei. Organisms associated with trichomoniasis include: Trichomonas vaginalis. The most common causes of vaginosis and vaginitis are: 1) proliferation of one or more anaerobic bacterial species in the vaginal tract leading to vaginal discharge without inflammation (22-50% of symptomatic women), known as bacterial vaginosis; 2) vulvovaginal candidiasis (17-39%); and 3) trichomoniasis (4-35%). Symptoms in undiagnosed women may be caused by a broad array of non-infectious conditions, including atrophic vaginitis, various vulvar dermatologic conditions, and vulvodynia. Abnormal vaginal discharge has a broad differential diagnosis, and successful treatment typically requires an accurate diagnosis.

2. Principles of the MVP Test

In one aspect, the MVP cartridge test is an automated in vitro diagnostic test for qualitative detection and differential diagnosis of DNA targets from anaerobic bacteria associated with bacterial vaginosis, Candida species associated with vulvovaginal candidiasis, and Trichomonas vaginalis, the agent of trichomoniasis. In an exemplary embodiment, the MVP cartridge is configured to be operated on a system that automates and integrates sample preparation, nucleic acid extraction and amplification, and detection of the target sequences in simple or complex samples using real-time PCR assays. Such systems can include an instrument, computer, and preloaded software for running tests and viewing the results. Such systems can utilize single-use disposable cartridges that hold the PCR reagents and host the PCR process. Because the cartridges are self-contained, cross-contamination between samples is minimized.

In an exemplary embodiment, the MVP cartridge includes reagents for the detection of DNA from BV organisms, Candida species, and Trichomonas vaginalis from vaginal swab samples. A Sample Processing Control (SPC) and a Probe Check Control (PCC) can also be included in the cartridge and utilized by the system instrument operating the cartridge. The SPC is present to control for adequate processing of the sample and to monitor for the presence of potential inhibitor(s) in the PCR reaction. The SPC also ensures that the PCR reaction conditions (e.g., temperature and time) are appropriate for the amplification reaction and that the PCR reagents are functional. The PCC verifies reagent rehydration, PCR tube filling, and confirms all reaction components are present in the cartridge including monitoring for probe integrity and dye stability.

In some embodiments, the MVP cartridge is designed for use with the following specimens collected from symptomatic individuals: self-collected vaginal swabs and clinician-collected vaginal swabs. The clinical specimens to be tested can be freshly obtained or in some embodiments, the specimen is preserved in a liquid solution. For example, a swab transport reagent included in the swab specimen collection kit is designed to collect and preserve patient specimens to allow transport to the laboratory prior to analysis with MVP cartridge test. In some embodiments, the swab sample is placed in the transport reagent within one hour, two hours, three hours, or six hours of the time the swab sample was collected. The transport reagent can be a commercially available reagent and can include saline, a carbonate, and a chelating agent. Using a transfer pipette, the sample is transferred to the sample chamber of the MVP cartridge. The cartridge is loaded onto the system platform, which performs hands-off, automated sample processing, and real-time PCR for the detection of DNA of the various targets associated with the panel. Typically, detailed test results are obtained within 90 minutes, within 75 minutes, within 60 minutes, within 45 minutes, within 30 minutes, or within 15 minutes and displayed in tabular and graphic formats.

The MVP Assay Cartridge can be provided within an MVP kit that contains sufficient cartridges with reagents to process multiple samples or quality control samples. Such kits can include: a set of MVP Cartridges (e.g. 10, 20, 40, 60, 80, 100, 120, etc.), a set of transfer pipettes (equal to or greater in number than cartridge set), and software instructions stored on a memory accessible by user (e.g. memory stick, CD, cloud) that cause the module to perform the MVP assay protocol. Table 1 below demonstrates MVP assay results and associated interpretations of an MVP Assay Panel performed by the cartridge described herein, and Table 2 further below shows an exemplary BV algorithm and expected results of an MVP cartridge test.

TABLE 1
MVP Results and Interpretations

| Result | Interpretation |
|---|---|
| BV NEGATIVE | Indicator DNA target(s) related to bacterial vaginosis (BV) organisms |
| Candida group | is/are not detected (see Table 2); Candida group (C. albicans and/or |
| NOT DETECTED | C. tropicalis and/or C. parapsilosis and/or C. dubliniensis) target |
| Candida | DNA is not detected; Candida glab-krus (Candida glabrata and/or |
| glab-krus | C. krusei) target DNA is not detected; and Trichomonas vaginalis (TV) |
| NOT DETECTED | target DNA is not detected. |
| TV NOT | SPC: PASS; SPC has a Ct within the valid range and endpoint |
| DETECTED | above the threshold setting. |
| | PCC: PASS; all probe check results pass. |
| BV POSITIVE | Indicator DNA target(s) related to bacterial vaginosis (BV) organisms |
| Candida | is/are detected (see Table 2); Candida group (C. albicans and/or |
| group | C. tropicalis and/or C. parapsilosis and/or C. dubliniensis) target DNA is |
| DETECTED | detected; Candida glab-krus (Candida glabrata and/or C. krusei) |
| Candida | target DNA is detected; and Trichomonas vaginalis (TV) target DNA |
| glab-krus | is detected. |
| DETECTED | BV, Candida group, Candida glab-krus, and TV: Ct values are |
| TV | within the valid range. |
| DETECTED | SPC: NA (not applicable); SPC signal is not part of the result |
| | interpretation algorithm if the target DNA is detected since SPC |
| | signal may be suppressed due to competition with BV, Candida |
| | group, Candida glab-krus, and TV targets. |
| | PCC: PASS; all probe check results pass. |
| INVALID | Presence or absence of the target DNA cannot be determined. |
| | BV, Candida group, Candida glab-krus, and TV: one or more of |
| | the analyte results is INVALID. |
| | SPC: FAIL or NA. |
| | PCC: PASS; all probe check results pass. |
| ERROR | Presence or absence of BV, Candida group, Candida glab-krus, and |
| | TV target DNA cannot be determined. |
| | BV, Candida group, Candida glab-krus, and TV: NO RESULT |
| | SPC: NO RESULT |
| | PCC: FAIL; all or one of the probe check results fail. |
| NO RESULT | Presence or absence of BV, Candida group, Candida glab-krus, and |
| | TV target DNA cannot be determined. A NO RESULT indicates |
| | that insufficient data were collected. For example, cartridge integrity |
| | test failed, the operator stopped a test that was in progress or a power |
| | failure occurred. |
| | BV, Candida group, Candida glab-krus, and TV: NO RESULT |
| | SPC: NO RESULT |
| | PCC: NA (not applicable) |

TABLE 2
Bacterial Vaginosis (BV) Results Algorithm^{a}

| BV Organisms | | | |
|---|---|---|---|
| | Megasphaera-1 | BVAB2 | |
| Atopobium spp. ^{b} | (Ct value within | (Ct value within | |
| (Ct value within the | the valid Ct | the valid Ct | |
| valid Ct range) | range) | range) | BV Result |
| + | + | − | BV Positive |
| + | − | + | BV Positive |
| + | + | + | BV Positive |
| + | − | − | BV Positive |
| (high concentration) | | | |
| − | +/− | +/− | BV Negative |

^{a}Algorithm results are either BV positive or BV negative.
^{b} Atopobium vaginae and/or Atopobium novel species.

In another aspect, assay workflows have been specifically developed for performing the multi-target assay panels with the sample cartridge described herein, in particular an MVP assay panel, such as the MVP 200 shown in FIG. 2A. FIG. 2B shows an example workflow 210 for the MVP. The workflow includes the steps of: receiving the sample 211; lysing the targets 212; binding nucleic acid to filter 213; optionally washing the filter 214; eluting the nucleic acid 215; optionally performing reverse transcription-PCR 216; and performing PCR 217. Steps 211 through 217 are performed within the cartridge body having the processing chambers therein, and the reaction vessel attached to the cartridge body. In exemplary embodiment, this workflow uses the MVP cartridge described herein, which relies on RNA/DNA capture and purification, target capture by affinity binding by use of a glass filter, preparation buffers to chemically lyse the multiple target organisms as well as create ideal conditions for nucleic acid binding, optional washing and elution. In some embodiments, the filter can include glass fibers and optionally a polymeric binder. In some examples, the glass fibers can be modified with a DNA binding ligand such as an alkylamine, a cycloalkylamine, an alkyloxy amine, a polyamine moiety, an arylamine, an intercalating agent, a DNA groove binder, a peptide, an amino acid, a protein, or a combination thereof. Examples of modified glass fibers that can be used in the compositions, systems, and methods disclosed herein are described in U.S. patent application Ser. No. 18/309,691 titled “NUCLEIC ACID EXTRACTION AND ISOLATION WITH HEAT LABILE SILANES AND CHEMICALLY MODIFIED SOLID SUPPORTS,” which is incorporated herein by reference in its entirety. In some embodiments, the filter can be a 500 microns to 2000 microns thick glass fiber disk having a pore size of 0.2 microns to 2 micron or from 0.4 microns to 1 micron.

### C. Example Valve Assemblies

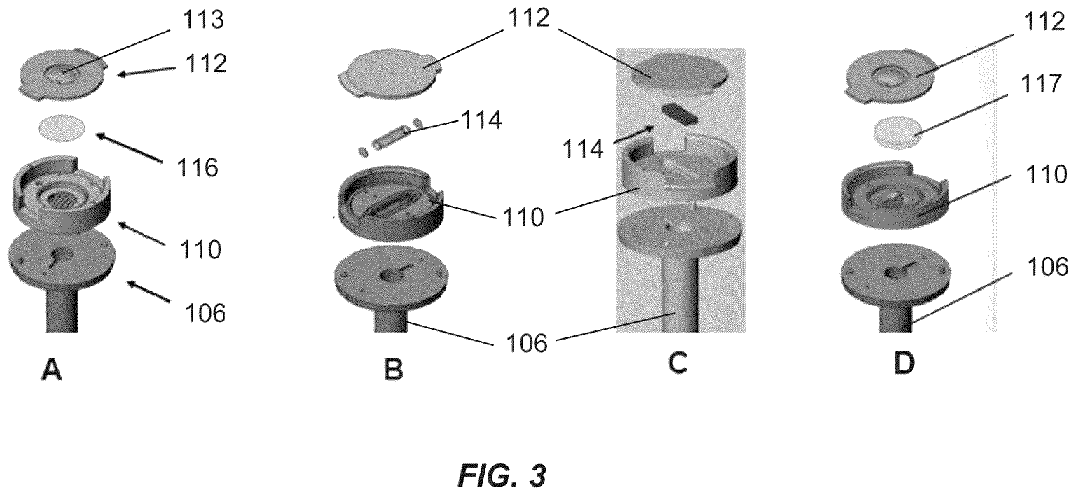

FIG. 3 illustrates differing valve assembles that can be used in the sample cartridge of FIG. 1A. Each of the valve assemblies is shown inverted to better illustrate the components of the valve body. Valve assembly A performs only mechanical lysing and is suitable for lysing hardy targets (e.g. certain bacteria, spores). Valve assemblies B and C perform only chemical lysing and is suitable for lysing less hardy targets (e.g. viruses, free NA, some spores, some bacteria and yeasts). Valve assembly D can perform both mechanical and chemical lysing for all types of targets. In all such cartridges, the valve assembly includes the syringe tube 106, valve body 110, and valve cap 112. In one aspect, the additional capabilities of the valve assembly of the sample cartridge rely in part on the filter, features of the valve body and cap, as well as the particular workflow sequence performed by the instrument interface of the module. For example, valve assembly A has a valve body 110 shaped with a circular cavity to support a polymer filter disc 116 to filter the sample, and the cap 112 has a sonication dome feature 113, which interfaces with a sonication horn of a cartridge receiving module so as to ultrasonically lyse the target. By contrast, valve assemblies B and C have a valve body 110 with an oblong filter recess 111 that receives a glass filter 114 (e.g. glass column) therein, the glass filter configured for binding with nucleic acid released from the target by chemical lysing. Valve assembly D has a design more similar to valve assembly A, having a cap 112 with a sonication dome 113, and a valve body with a circular cavity for supporting a disc filter, however this design uses a glass fiber filter 117. Utilizing glass fibers to form the filter facilitates affinity bonding with the free nucleic acid released by chemical lysing. Thus, this filter is suited for both mechanical and chemical lysing. U.S. Application Publication No. 2023/0044516A1 further details the assay cartridge of valve assembly D and is incorporated herein by reference in its entirety.

### D. Reaction Modules

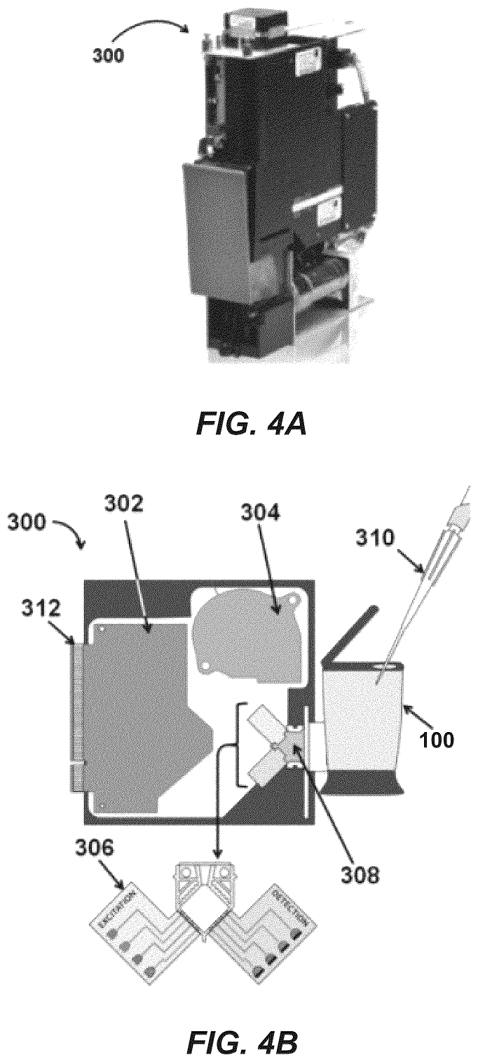

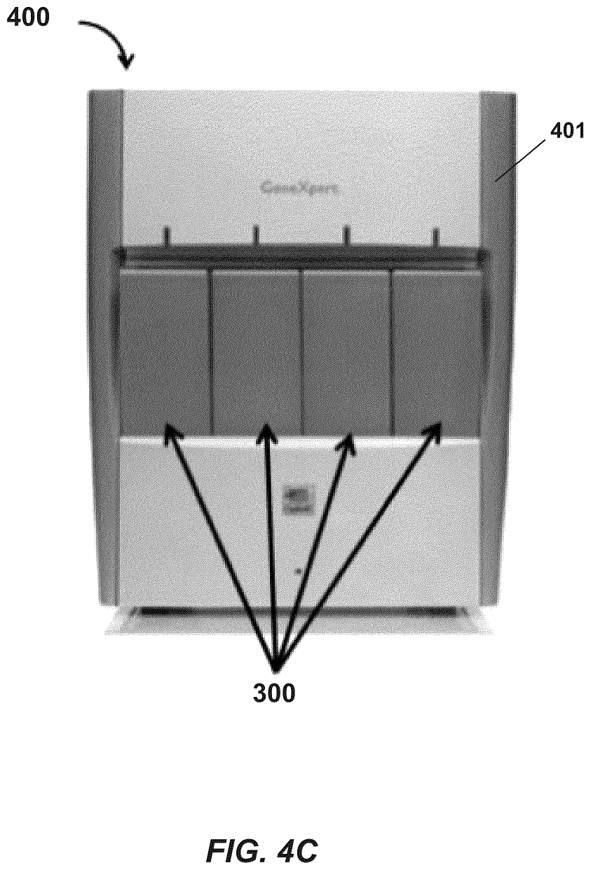

In certain embodiments the cartridge 200 is configured for insertion into a reaction module 300, e.g., as shown in FIG. 4A. As illustrated in FIG. 4B the module is configured to receive the cartridge 200 therein. In certain embodiments the reaction module provides heating plates 308 to heat the temperature controlled chamber or channel. The module can optionally additionally include a fan 304 to provide cooling where the temperature controlled channel or chamber is a thermocycling channel or chamber. Electronic circuitry 302 can be provided to pass information (e.g., optical information) top a computer for analysis. In certain embodiments the module can contain optical blocks 306 to provide excitation and/or detection of one or more (e.g., 1, 2, 3, 4, or more) optical signals representing, e.g., signal DNAs amplified for various PCR targets. In various embodiments an electrical connector 312 can be provided for interfacing the module with a system (e.g. system controller or with a discrete analysis/controller unit. As illustrated, in FIG. 4B the sample can be introduced into the cartridge using a pipette 310. In certain embodiments, the module also contains a controller that operates a plunger in the syringe barrel and the rotation of the valve body.

### E. Analytical System

In certain embodiments a system (e.g., a processing unit) is provided. One illustrative, but non-limiting embodiment is shown in FIG. 4C. System 400 includes an enclosure 401 that is configured to support and power multiple sample processing modules 300, where each processing module is configured to hold and operate a removable cartridge 100. In some embodiments, the system is configured to operate the sample processing modules to perform a PCR assay for one or more target analytes and optionally to determine the level of one or more target RNA/DNA sequences within a corresponding removable sample cartridge. Typically, the processing on a sample within the corresponding removable sample cartridge involves operating the cartridge to perform a method as described herein. In certain embodiments the system is configured to contain one sample processing module. In certain embodiments the system is configured to contain at least two or more sample processing modules (e.g., at least 4, 8, 12, 16, 20, 24, 28, 32, 64, 128 or more) sample processing modules. In some embodiments, the system provides a user interface that allows the user input operational instructions and/or to monitor operation of the cartridges to determine the presence and/or quantity of one or more nucleic acids.

While the methods described herein are described primarily with reference to the GENEXPERT® cartridge by Cepheid Inc. (Sunnyvale, Calif.) (see, e.g., FIG. 1A), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1 and KR102362853B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Such cartridge/microfluidic systems can include, for example microfluidic systems implemented using soft lithography, micro/nano-fabricated microfluidic systems implemented using hard lithography, and the like.

In one exemplary embodiment, the cartridge can include a plurality of cartridge bodies, such as a first body, a second body, a central syringe barrel that is in fluid communication with the first body and the second body, a reaction vessel, and the like. The first body may be formed of a plurality of chambers separated from each other for reagents or buffers and sample processing. In some embodiments, the first body can be used for the purpose of storing a plurality of reagents. The second body may be formed of one or a plurality of chambers separated from each other and includes a path through which the reagent or sample from the first body moves. When the first body and the second body of cartridge are combined, a liquid flow path and optionally an air flow path can be formed between both compartments via the central syringe barrel. The liquid flow path is connected to the first body to provide a space for samples and reagents to move and mix. The air flow path may connect the reaction vessel and a vacuum control region of the “plunger” to control the vacuum that may occur when the extracted nucleic acid moves to the reaction vessel. Rotation of the syringe barrel comprising a “plunger” that can sequentially suck sample and reagents from the plurality of chambers into an interior space of the syringe barrel, and discharge the mixture of the interior space into any one of the plurality of chambers (first body or second body) of the cartridge. Rotation of the syringe barrel comprising a “plunger” can suck the reagent inside the plurality of chambers of the cartridge into the interior space of the syringe barrel and then discharge the mixed reagent to a nucleic acid amplification reaction vessel.

In another exemplary embodiment, the cartridge includes a flow cover and a base plate, which together form a closed passage therein. In one embodiment of this configuration, an inner chamber containing the reagents required for dielectric extraction is provided separately from an outer chamber, and the upper and lower portions of the inner chamber are sealed. In addition, a double-structured flow cover-pad can be disposed between the outer chamber and the base plate. Closed flow paths are formed by achieving a strong coupling between the base plate—the flow cover—the pad—the outer chamber. Also provided in this configuration are beads necessary for dielectric extraction and amplification which are accommodated in a dual chamber structure of an outer chamber-bead chamber. The beads can be maintained by a dehumidifying unit positioned above the bead chamber even when the bead chamber is opened.

In further exemplary embodiment, the cartridge can include a plurality of reaction chambers, particularly, the reaction vessel can include a plurality of reaction chambers. In these embodiments, different types of lyophilized primers and probes can be provided in each reaction chamber. For example, primers and probes for BV associated nucleic acids can be provided in one reaction chamber, and primers and probes for Candida spp. amplification and detection can be provided in a second chamber, and such the like. Of course, it is possible to perform various amplification and detection processes at the same time in a single reaction chamber. Accordingly, amplification and detection of each target described herein may be performed individually in separate reaction chambers or wells, or carried out in a multiplex reaction in a single reaction chamber or well.

Additionally, it is appreciated that the assay panel methods described herein (i.e., identification of multiple conditions based on comparative levels of multiple-targets obtained from a single sample) can further be realized in entirely different systems, including: isothermal nucleic acid amplification systems, digital RT-PCR, electrochemical PCR, lateral flow testing cartridges, electrochemical sensors, nucleic acid sequencing, CRISPR/Cas based technologies, chemiluminescence, and nanoparticle-based colorimetric detection.

In various embodiments, the signal DNA(s) from PCR (nucleic acid amplification) reactions are amplified for detection and/or quantification. In certain embodiments, the amplification comprise any of a number of methods including, but not limited to polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), rolling circle amplification (RCA), and the like.

In illustrative, but non-limiting embodiments, the amplification reaction may produce an optical signal that is proportional to the amount of amplified target nucleic acid (e.g., signal DNA). Illustrative optical signals include, but are not limited to a fluorescent signal, a chemiluminescent signal, an electrochemiluminescent signal, a colorimetric signal, and the like. In certain embodiments the optical signal is a fluorescent optical signal generated by a fluorescent indicator. In certain embodiments the fluorescent indicator is a non-specific intercalating dye that binds to double-stranded DNA products, while in certain other embodiments, the fluorescent indicator comprises a target sequence specific probe (e.g., a TAQMAN® probe, a SCORPION® probe, a MOLECULAR BEACON®, and the like).

Single PCR reactions (nucleic acid amplification), or multiple PCR reactions (nucleic acid amplifications) run sequentially (or simultaneously in separate temperature controlled channels or chambers) can also use the same detectable label since sequentially run PCR signal DNAs are analyzed sequentially and the simultaneous PCR signal DNAs are distinguished by the occurrence in different temperature controlled channels or chambers. The signal produced by this amplification can be distinguished from other amplification products because it is not run at the same time and/or because it is run in a different reaction channel/chamber. However, where multiple nucleic acid amplifications are run simultaneously in the same chamber the reaction products of for each analysis are typically detected and/or quantified by the use of different and distinguishable labels.

In certain embodiments, amplification products (amplified nucleic acid from nucleic acid analysis) can be detected using methods well known to those of skill in the art. In certain embodiments the amplification is a straightforward simple PCR amplification reaction. In certain embodiments, however, a nested PCR reaction is used to amplify the nucleic acid from the nucleic acid analysis. In various embodiments, multiplexed PCR assays are contemplated, particularly where it is desired to analyze multiple products of the nucleic acid analysis in the same amplification reaction. In certain embodiments in such multiplexed amplification reactions, each probe (e.g., for each specific analyte) has its own specific dye/fluor so that it is detectable independently of the other probes. In certain embodiments, typically, for signal generation, the probes used in various amplification reactions utilize a change in the fluorescence of a fluorophore due to a change in its interaction with another molecule or moiety brought about by changing the distance between the fluorophore and the interacting molecule or moiety for detection and/or quantification of the amplified product. Alternatively, other methods of detecting a polynucleotide in a sample, including, but not limited to, the use of radioactively-labeled probes, are contemplated.

### III. Assay Workflows

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g. DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. For example, sample preparation can include heating the sample in a lysis solution without further purification prior to carrying out the amplification reaction. In some embodiments, the lysed sample may be diluted prior to carrying out the amplification reaction. One or more of these various sample preparation operations are readily incorporated into the fluidly closed cartridge systems contemplated herein.

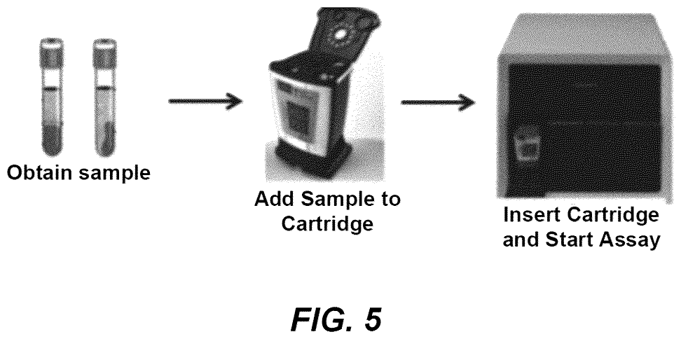

FIG. 5A shows illustrative hut non-limiting workflows for nucleic acid amplification. In one aspect, the assay sample cartridge, as described herein, is capable of a specialized workflow that performs lysing and detection of differing target analytes as required for a particular assay panel. In some embodiments, the cartridge is configured for chemical lysing of the multiple targets. In other embodiments, the cartridge is configured for mechanical lysing of the multiple targets. In still other embodiments, the cartridge is configured for both mechanical and chemical lysing to allow lysing of multiple targets of differing types. Accordingly, the sample cartridge can be configured to perform the assay panel by an existing workflow associated with conventional cartridges, or can be operated according to an new workflow specially configured for the assay panel.

In one aspect, the sample cartridge having a valve assembly as described in FIG. 3D herein, is capable of a variety of workflows that perform: chemical lysing of targets, mechanical lysing of targets, or both. Accordingly, the sample cartridge can perform an existing workflow associated with conventional specialized cartridges, or can perform entirely new workflows that perform both.

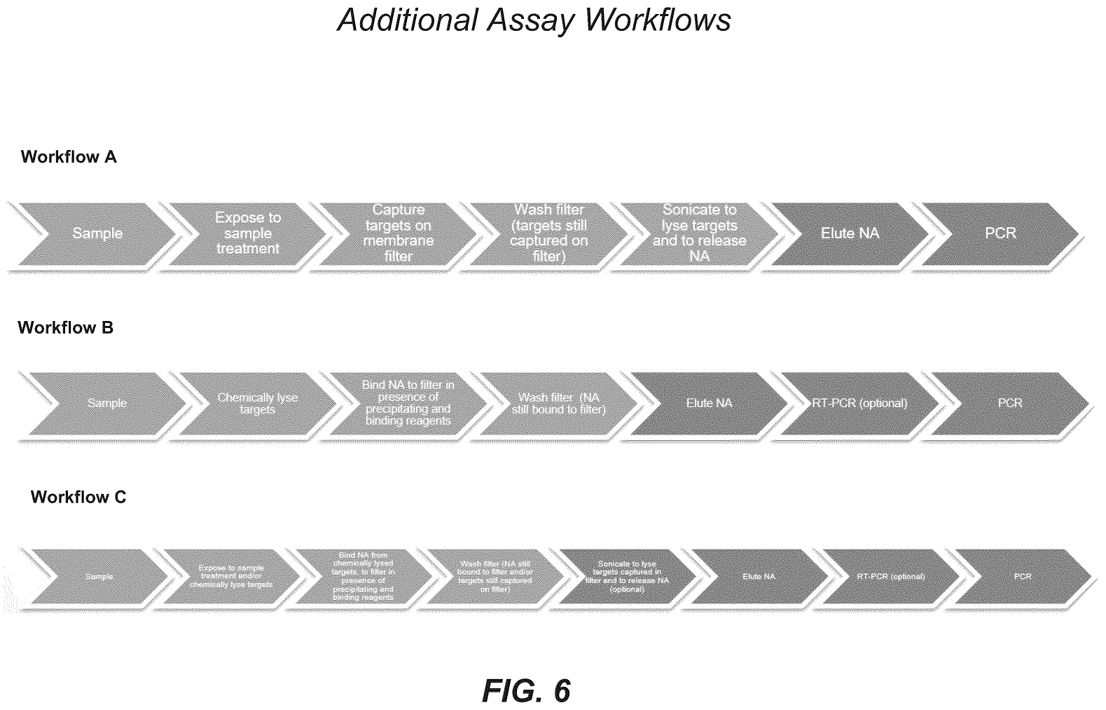

Exemplary assay workflows that can be performed with a single universal cartridge, in accordance with some embodiments, are shown in FIG. 6. In any of these embodiments, the filter can be formed of glass filter to promote affinity binding of the nucleic acids (NA) to the glass fibers and a pore size suited for chemical lysing as well. In any of these workflows, the nucleic acid amplification can be PCR, real-time PCR, isothermal amplification (including but not limited to nucleic acid sequence-based amplification, loop-mediated isothermal amplification, helicase-dependent amplification, rolling circle amplification, multiple displacement amplification, whole genome amplification or recombinase polymerase amplification) or other nucleic acid amplification methods known to persons of skill in the art.

In Workflow A, the sample is optionally exposed to a sample treatment or chemically lysed, then the treated or lysed fluid sample is flowed through the filter where targets are captured. In some embodiments, the sample treatment is used to either weaken the cell wall or to inactivate the sample or make it less viscous to facilitate being processed through the filter. The filter is then washed, leaving the targets on the filter. Next, the targets are mechanically lysed, such as by sonication, to release nucleic acid (NA). In some embodiments, mechanical lysing includes in-filling glass beads along the filter to aid in mechanical lysing of the target. Next, the NA is eluted from the filter and then nucleic acid amplification is performed is performed.

In Workflow B, the sample is chemically lysed to obtain the NA targets. In some embodiments, after chemically lysing, the NA is bound to the filter by the presence of precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the filter is eluted to remove the NA targets. In some embodiments, the elution is performed with a pH neutral buffer or basic buffer fluid. The target NA is then delivered to an attached reaction vessel to perform nucleic acid amplification.

In Workflow C, the fluid sample is exposed to sample treatment and/or chemically lyse the targets. Next, the NA freed by chemical lysing is bound to the filter. This step may utilize precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the targets captured in the filter are heat and/or mechanically lysed. This may utilize sonication, and may further utilize glass beads to facilitate mechanical lysing of select targets. Then, the lysed target NA is eluted from the filter. In some embodiments, the elution is performed with a pH neutral buffer or basic buffer fluid. The target NA is then delivered to an attached reaction vessel to perform nucleic acid amplification. Thus, in this workflow, the workflow allows for lysing of multiple differing targets, some requiring only chemically lysing (e.g. viral targets), and others requiring mechanical lysing (e.g. bacteria, spores, etc.), such that all these target NAs can be released from a single sample and tested by the same sample cartridge. While the above workflow described mechanical lysing after chemical lysing, it is appreciated that other workflows may be utilized in which chemical lysing occurs after mechanical lysing.

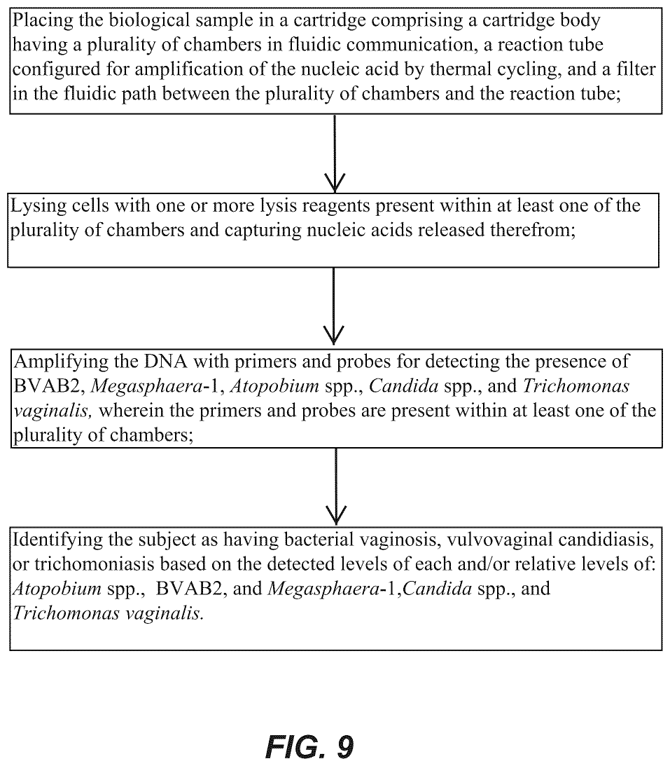

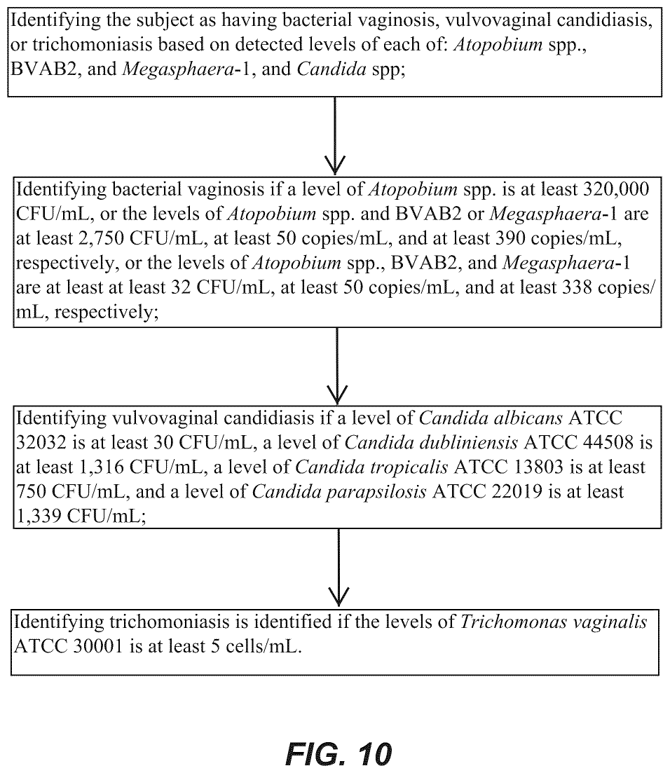

In some embodiments, the sample cartridge includes an identifier with information as to the appropriate workflow needed for a particular panel of assays, so that an instrument module receiving the sample cartridge operates according to the specified workflow. FIGS. 9-10 illustrate example methods of identifying a patient as having bacterial vaginosis, vulvovaginal candidiasis, or trichomoniasis, which can utilize the MVP cartridges described herein.

### IV. Multi-Target Panel Assay Configurations

### A. Reagents for Multi-Target Assay Panel

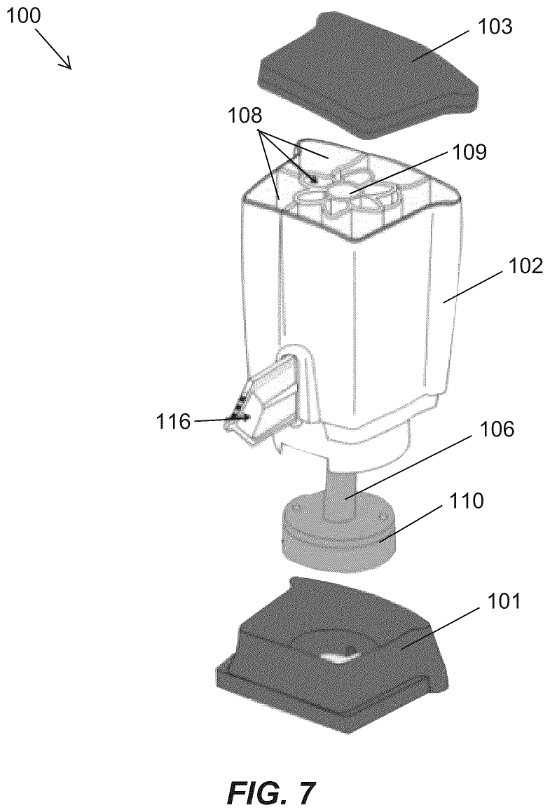

FIG. 7 shows an exemplary sample cartridge with the various chambers designated by number. The exemplary MVP cartridge can include: lyophilized reagents in the form of one or more beads as described herein; lysis reagent; alkaline agent, binding reagent, wash reagent, and eluting reagent.

In one aspect, the lysis reagent can include a chaotropic agent, a chelating agent, a buffer, an alkaline agent, or a detergent. The chaotropic agent can be selected from a guanidinium compound such as guanidinium thiocyanate or guanidinium hydrochloride, an alkali perchlorate such as lithium perchlorate, an alkali iodide, magnesium chloride, urea, thiourea, a formamide, or a combination thereof. The concentration of the chaotropic agent can range from about 1 M to about 10 M, such as from about 2.5 M to about 7.5 M. The chelating agent can be selected from N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. The concentration of the chelating agent can range from about 10 mM to about 100 mM and/or comprises about 0.5% to about 5% of the lysis reagent. The buffer can be selected from the group consisting of Tris, phosphate buffer, PBS, citrate buffer, TAPS, Bicine, Tricine, TAPSO, HEPES, TES, MOPS, PIPES, Cacodylate, SSC, and IVIES. The concentration of the buffer can range from about 5 mM to about 100 mM, such as from about 5 mM to about mM. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, and n-heptyl-β-D-glucopyranoside. The detergent can comprise about 0.1% to about 2% of the lysis reagent, and/or ranges from about 10 mM up to about 100 mM. The lysis reagent can have a pH ranging from about pH 3.0 to about pH 5.5.

In another aspect, the alkaline agent can be selected from an alkali metal hydroxide, such as sodium hydroxide or potassium hydroxide. The concentration of the alkaline agent can be about 0.5 N to 5 N.

In yet another aspect, the binding reagent can include a binding polymer such as polyacrylic acid (PAA), polyacrylamide (PAM), polyethylene glycol (PEG), poly(sulfobetaine), or combinations thereof. In some embodiments, the binding reagent and the wash reagent can be combined. For example, the binding reagent and/or the wash reagent can include a binding polymer (e.g., PEG 200), buffer, inorganic salt, antioxidant and/or chelating agent, antifoam SE15, sodium azide, disaccharides or disaccharide derivatives, carrier proteins, detergents, or DMSO. The binding polymer can be present in an amount of at least 10% v/v, at least 20% v/v, at least 30% v/v, or from 10% to 60% v/v, of the binding reagent or the wash reagent. The buffer can be selected from the group consisting of Tris, 2-amino-2-hydroxymethyl-1,3-propanediol, HEPES, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The salt, such as NaCl, KCl, or MgCl₂, can be present at a concentration from about 0.05 M to about 1 M, such as from about 0.1 M to about 0.5 M. The antioxidant and/or chelating agent comprises an agent selected from the group consisting of N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. In some embodiments the antioxidant and/or chelating agent comprises EDTA. In certain embodiments the antioxidant and/or chelating agent comprise 0.2% to about 5%, about 0.2% to about 3%, or about 0.5% to about 2%, or about 0.5% of the binding reagent or the wash reagent. In some embodiments the concentration of the antioxidant and/or chelating agent in the binding reagent or the wash reagent ranges from about 2 mM to about 50 mM or about 5 mM to about 20 mM. In some embodiments, the detergent is an ionic detergent or a non-ionic detergent. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, Brij-35, and n-heptyl-β-D-glucopyranoside. In some instances, to reduce bubble formation in one or more of the chambers, Brij may be added to the buffers (e.g., sample transport reagent, lysis reagent, binding reagent, wash reagent, or amplification reagent). The detergent can comprise about 0.1% to about 2% of the binding reagent or the wash reagent, and/or ranges from about 10 mM up to about 100 mM. The binding reagent and/or the wash reagent can have a pH ranging from about pH 6.0 to about pH 8.0 (such as from about 6.5 to about 7.5). It is understood that various other reagents and initial volumes can be used for performing an automated PCR assay panel on a sample inserted into the cartridge.

### B. Specialized Cartridge Components

FIG. 7 illustrates an exemplary cartridge suitable for performing a multi-target panel assay, as described herein. The illustrated cartridges are based on the GENEXPERT® cartridge (Cepheid, Inc., Sunnyvale, Calif.). The cartridge 100 comprises a cartridge body 102 having multiple chambers 108 defined therein for holding various reagent and/or buffers. The chambers are disposed around a central syringe barrel 109 that is in fluid communication with valve body 110 through valve syringe tube 106 extending through the syringe barrel 109. In some embodiments, the valve assembly include any of the valve assemblies described herein, or any suitable valve assembly. In this embodiment directed to an MVP cartridge, the cartridge utilizes a valve assembly capable of chemical lysing, for example, any of valve assemblies B-D in FIG. 3. The valve body 110 is interface within the cartridge body and supported on a cartridge base 101. The cartridge typically contains one or more channels or cavities that can contain a filter material (e.g. glass filter column) as described herein that can function to bind and elute a nucleic acid. In various embodiments, the cartridge further comprises one or more temperature controlled channels or chambers that can, in certain embodiments, function as thermocycling chambers. A “plunger” not shown can be operated to draw fluid into the syringe barrel 109 and rotation of the valve body/syringe tube provides selective fluid communication between the various reagent chambers and channels, reaction chamber(s). Thus, the various reagent chambers, reaction chambers, matrix material(s), and channels are selectively in fluid communication by rotation of the valve and plunger and reagent movement (e.g., chamber loading or unloading) is operated by the “syringe” action of the plunger. The attached reaction vessel 116 provides optical windows to provide real-time detection of, e.g., amplification products, base identity in sequencing operations, by operation of the module within the system described herein.

While the methods described above are described with respect to specific chambers in the GENEXPERT® cartridge, it will be recognized that the particular reagent/chamber assignments can be varied depending on the particularities of the nucleic acid detection/quantification assay. It will also be recognized that in certain embodiments, variants of the GENEXPERT® cartridge are also contemplated. Such variants can include, but are not limited to, more reagent chambers or fewer reagent chambers and/or different sized chambers, two (or more) sample receiving chambers, two (or more) temperature controlled channels or chambers, stacked cartridges (providing control of two cartridges by one module), and the like. In one aspect, the sample cartridge includes one or more features or components that are specially configured per the unique requirements of a particular multi-target assay. In this embodiment, the sample cartridge utilizes certain components specifically developed for the MVP assay.

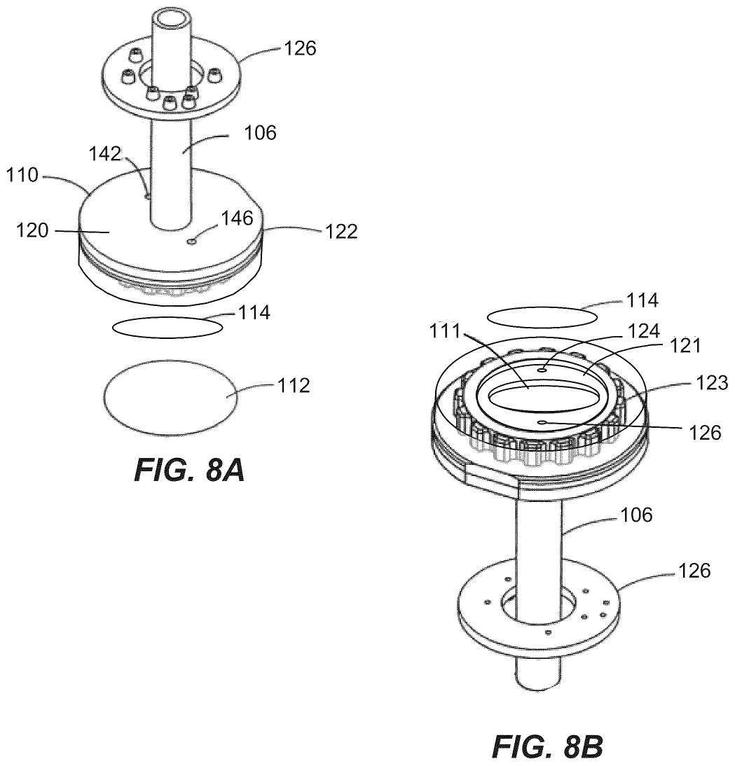

FIGS. 8A-8B show a detailed view of a valve assembly for use in a multi-target assay panel cartridge, in accordance with some embodiments. In this embodiment, the valve assembly is configured for chemical lysing. The valve assembly includes a valve body 110 having a disk shape and tubular syringe type 106 along a proximal portion thereof. The disk portion has a generally planar external upper port surface 120 having one or more ports therein (e.g. 142, 146), as shown in FIG. 8A. The valve assembly is rotatable relative to the cartridge housing, for example by engagement of an interface feature 123 with a driven mechanism of the module. The ports 142, 146 on the top planar surface permit fluid communication between the syringe tube 106 and each of the chambers within the cartridge and/or one or more channels. In this embodiment, the ports 142, 146 interface with a circular gasket 126 having multiple ports defined therein which interfaces and fluidically couple with corresponding ports on the bottom of the chambers in the cartridge body. Upon rotation of the valve assembly, the top ports 142, 146 interface with differing pairs of ports so as to fluidically couple the various chambers to each other or to channels of the reaction vessel attached to the cartridge so that movement of the “plunger” through the syringe tube advances fluid between the various chambers and/or channels to facilitate processing and subsequent analytical testing of the fluid sample.

As seen in FIG. 8B, the valve body 110 includes a lysing chamber 121 on an underside thereof that may contain solid phase material for capturing cells, spores, viruses, or microorganisms to be lysed. Suitable solid phase materials include, without limitation, filters, beads, fibers, membranes, filter paper, glass wool, polymers, or gels. One or more interior ports facilitate fluid flow into and out of the lysing chamber. In this embodiment, the solid phase material is a glass filter 114 that resides in a filter recess 111 in the valve body 110 for capturing nucleic acid released by chemical lysing of the target analytes in the fluid sample.

As shown in FIGS. 8A-8B, an outer wall 122 of the valve body encloses the lysing chamber 121 along with bottom cap 112. As seen in FIG. 8B, the lysing chamber 121 includes a pair of processing ports to facilitate flow of fluid sample into and out of the lysing chamber. A first fluid processing port 124 coupled to a first fluid processing channel defined within the valve body, and a second fluid processing port 125 coupled to a second fluid processing channel defined within the valve body. Through these channels, the first fluid processing channel is coupled to both the inner conduit of the syringe tube 106 and external port 142, while the second fluid processing channel is coupled to the second external port 146. By this configuration, application of pressure by movement of a “plunger” through the syringe tube 106 acts like a syringe, drawing fluid from one chamber into the syringe tube, and after rotation of the valve assembly to position the respective ports to couple with another chamber, movement of the plunger advances the fluid through the valve body and channels into another chamber or channel to the reaction vessel. This approach can perform the various processing steps required to perform a sample preparation protocol for the targeted analytes of the multi-target assay panel.

In one aspect, the MVP discussed above performs chemical lysing of the targeted bacteria, protozoa and yeasts. Often, in conventional sample cartridges and methods, these bacteria, protozoa and yeast targets are mechanically lysed (e.g. by ultrasonic lysing), whereas chemical lysing is usually reserved for less robust target such as viruses. In order to perform chemical lysing of these targets, lysing buffers have elevated alkalinity (e.g. sodium hydroxide) are required. Further high alkalinity eluting buffers (e.g. ammonia or an alkali metal hydroxide) may be used to elute the nucleic acids bound to the glass filter. While these buffers allow for chemical preparation of the sample, in practice, use of such buffers is problematic in conventional cartridge as these high alkalinity buffers can degrade the valve assembly material and sealing interfaces between cartridge components, resulting in cracking of the valve assembly and leakage during processing. Such leakage is detrimental to sample processing, such that specialized components were developed to address this unique problem associated with the MVP cartridge. A specialized valve assembly was developed to resist elevated alkalinity (e.g., greater than pH of 10, greater than pH 11, or greater than pH 12) of these buffers. One difficulty in developing these valve assemblies, such as that in FIGS. 8A-8B, is that these valve assemblies are fabricated by injection molding of certain polymer materials, typically polycarbonate, polyolefin (including polyethylene or polypropylene), or combinations thereof. Typical such polymer materials used in injection molding are not resistant to the range of elevated alkalinity noted, and polymer materials that are resistant, may be considerably more costly and may be less suited for injection molding of small-scale microfluidic features. Thus, the specialized valve assemblies were developed by annealing valve body assemblies formed with conventional polymers in order to harden the polymer material sufficiently to resist elevated alkalinity. Annealing of polycarbonate reduces chemical corrosion by the mixture of NaOH and GTC within the lysis reagent and the eluting reagent. In some embodiments, after the valve assemblies are formed, they are heated to an elevated temperature (e.g. 90-100° C., about 100° C.) for about an hour or more, then allowed to slowly cool in a temperature-controlled manner for at least 30 minutes. Studies showed that these annealed valve assemblies resulted in valve assemblies of substantially the same design and material as conventional valve assemblies, except they were resistant to high alkalinity buffers that could not have been feasible to use in conventional cartridges. After annealing of the valve body assembly is performed, testing by exposing VBA to NaOH/GTC is performed and the valve assembly is visually inspected for cracking of VBA after min or so. Thus, by use of these specialized features, the sample cartridge can perform chemical lysing of the target yeasts, protozoa, and bacteria of the MVP that would otherwise not be feasible. It is appreciated that this annealed cartridge can be advantageous for various reasons and need not be tied to any particular assay described herein. Moreover, it is further appreciated that the assays described herein may be performed with various other cartridge designs, devices and systems and need not be tied to the particular cartridge designs described herein.

### V. Assay Methods

Disclosed herein are method for detecting or differentially diagnosing bacterial vaginosis, vulvovaginal candidiasis/vulvovaginal candidiasis-associated species/Candida spp., trichomoniasis/trichomoniasis-associated species/Trichomonas vaginalis, or a combination thereof in a biological sample obtained from a subject. The subject is typically a mammal, wherein the mammal is typically a human. The method can include steps essentially of contacting nucleic acid from a biological sample from the subject with sets of primers and probes that amplify and detect BVAB2, Megasphaera-1, Atopobium spp., Candida spp., and Trichomonas vaginalis, and conducting one or more polymerase chain reaction (PCR); and detecting an amplicon that is produced by the PCR. The method can include identifying the subject as having bacterial vaginosis, vulvovaginal candidiasis/vulvovaginal candidiasis-associated species/Candida spp., trichomoniasis/trichomoniasis-associated species/Trichomonas vaginalis, or a combination thereof based on detected levels of each of: Atopobium spp., BVAB2, and Megasphaera-1, Candida spp. and Trichomonas vaginalis.

In some embodiments, the primers and/or probes for amplifying and detecting the presence of Atopobium vaginae comprise or consist essentially of a sequence that is identical or complementary to at least 15 contiguous nucleotides of one or more of SEQ ID NOs: 1, 2, and 3; the primers and/or probes for amplifying and detecting the presence of BVAB2 comprise a sequence that is identical or complementary to at least 15 contiguous nucleotides of one or more of SEQ ID NOs: 4, 5, and 6; the primers and/or probes for amplifying and detecting the presence of Megasphaera-1 comprise a sequence that is identical or complementary to at least 15 contiguous nucleotides of one or more of SEQ ID NOs: 7, 8, and 9; the primers and/or probes for amplifying and detecting the presence of Candida spp. (Candida albicans/dubliniensis/tropicalis/parapsilosis) comprise a sequence that is identical or complementary to at least 15 contiguous nucleotides of one or more of SEQ ID NOs: 10, 11, 12, 13, 14, 15, and 16; the primers and/or probes for amplifying and detecting the presence of Candida glabrata comprise a sequence that is identical or complementary to at least 15 contiguous nucleotides of one or more of SEQ ID NOs: 17, 18, and 19; the primers and/or probes for amplifying and detecting the presence of Candida krusei comprise a sequence that is identical or complementary to at least 15 contiguous nucleotides of one or more of SEQ ID NOs: 20, 21, and 22; and the primers and/or probes for amplifying and detecting the presence of Trichomonas vaginalis comprise a sequence that is identical or complementary to at least 15 contiguous nucleotides of one or more of SEQ ID NOs: 23, 24, and 25. The oligonucleotide sequences are shown in Table 3. The amplicons that would be generated for each target are shown in Table 4.

TABLE 3
Target Primers and Probes

| | | | | SEQ |
|---|---|---|---|---|
| | | | | ID |
| Target | Primer | Sequence | Length | NOs |
| Atopobium | Forward | CTTCGGATGTGTATAAAGTGGCG | 23 | 1 |
| vaginae | Reverse | AGGTTAATACCGGATACTCCATAT | 24 | 2 |
| | Probe | TTTGCACTGGGATAGCCTCGGGA | 23 | 3 |
| Megasphaera- | Forward | GCGTGAGTGAAGAAGGTCTTCGGA | 24 | 4 |
| 1 | Reverse | CGAAAAAGACGGATGCCAACAGTATCCGT | 21 | 5 |
| | Probe | GCTAACTACGTGCCAGCAGCC | 29 | 6 |
| BVAB2 | Forward | GCGGCTAGATAAGTGTGATGTTT | 23 | 7 |
| | Reverse | GCTGGAGAGGATAGTGGAATTCCTAGTG | 28 | 8 |
| | Probe | CCTTGGGGTTCATTACAAACTGTTTAGCT | 29 | 9 |
| Candida | Forward | GCTTTAGCAGCAATAATGTGTTCAAC | 26 | 10 |
| albicans/ | | GCCTTAGCAGTGATGATGTGTTCAACCA | 28 | 11 |
| dubliniensis/ | Reverse | ATTTTACCAGCATCTCTGTATTTAG | 25 | 12 |
| tropicalis/ | Probe | TGTTACCTTTAGCAGCTTTGTACAAGTT | 28 | 13 |
| parapsilosis | | | | |
| Candida | Forward | TTGGCATAAAGGACCCGTAGG | 21 | 14 |
| albicans | Reverse | CGATAGAATCTATTAGCTGGTAGTATGTA | 29 | 15 |
| | Probe | TGTGTATTTAATAAGATAGCCCGTCCTCCA | 33 | 16 |
| | | GAG | | |
| Candida | Forward | GAGGCCGACTACACGACGACGAT | 23 | 17 |
| glabrata | Reverse | ATCCCATTGAAGCCAAGTGATGCCG | 25 | 18 |
| | Probe | AAGGACTCTGACGGCAAGCCTA | 22 | 19 |
| Candida | Forward | GGACATTCAGATAATCCAGATTTTTCAAAG | 30 | 20 |
| krusei | Reverse | TGGACAAATATCAGCTAATAAAATGA | 26 | 21 |
| | Probe | AAACATCGAATCCCTTACCCGTGAAGAA | 28 | 22 |
| Trichomonas | Forward | GTAACAACCTTGGAGTTCTTCTTAAG | 26 | 23 |
| vaginalis | Reverse | TCAAGTGGTGTCTTGTAGATTGATGT | 26 | 24 |
| | Probe | AGTTTGGCTGCTTAGCTTCGAC | 22 | 25 |

In some embodiments, at least one of the primers and/or probes for amplifying and detecting the presence of Atopobium vaginae comprise or consist essentially of a sequence that is identical or complementary to at least 10 (at least 12, at least 14, or at least 15) contiguous nucleotides of SEQ ID NO: 26; at least one of the primers and/or probes for amplifying and detecting the presence of BVAB2 comprise a sequence that is identical or complementary to at least 10 (at least 12, at least 14, or at least 15) contiguous nucleotides of one or more of SEQ ID NO: 27; at least one of the primers and/or probes for amplifying and detecting the presence of Megasphaera-1 comprise a sequence that is identical or complementary to at least 10 (at least 12, at least 14, or at least 15) contiguous nucleotides of SEQ ID NO: 28; at least one of the primers and/or probes for amplifying and detecting the presence of Candida albicans comprise a sequence that is identical or complementary to at least 10 (at least 12, at least 14, or at least 15) contiguous nucleotides of SEQ ID NO: 29 or 30; at least one of the primers and/or probes for amplifying and detecting the presence of Candida dubliniensis comprise a sequence that is identical or complementary to at least 10 (at least 12, at least 14, or at least 15) contiguous nucleotides of SEQ ID NO: 31; at least one of the primers and/or probes for amplifying and detecting the presence of Candida tropicalis comprise a sequence that is identical or complementary to at least 10 (at least 12, at least 14, or at least 15) contiguous nucleotides of SEQ ID NO: 32; at least one of the primers and/or probes for amplifying and detecting the presence of Candida parapsilosis comprise a sequence that is identical or complementary to at least 10 (at least 12, at least 14, or at least 15) contiguous nucleotides of SEQ ID NO: 33; at least one of the primers and/or probes for amplifying and detecting the presence of Candida glabrata comprise a sequence that is identical or complementary to at least 10 (at least 12, at least 14, or at least 15) contiguous nucleotides of SEQ ID NOs: 34; at least one of the primers and/or probes for amplifying and detecting the presence of Candida krusei comprise a sequence that is identical or complementary to at least 10 (at least 12, at least 14, or at least 15) contiguous nucleotides of SEQ ID NOs: 35; and at least one of the primers and/or probes for amplifying and detecting the presence of Trichomonas vaginalis comprise a sequence that is identical or complementary to at least 10 (at least 12, at least 14, or at least 15) contiguous nucleotides of SEQ ID NO: 36.

TABLE 4
Target MVP Amplicon Sequences

| | | Amplicon | SEQ ID |
|---|---|---|---|
| Target | Sequence | size (nt) | NOs |
| Atopobium | CTTCGGATGTGTATAAAGTGGCGAACGGCTG | 106 | 26 |
| vaginae | AGTAACACGTGGGCAACCTGCCCTTTGCACT | | |
| | GGGATAGCCTCGGGAAACCGAGGTTAATAC | | |
| | CGGATACTCCATAT | | |
| Megasphaera- | GCGTGAGTGAAGAAGGTCTTCGGATTGTAA | 126 | 27 |
| 1 | AACTCTGTTATACGGGACGAAAAAGACGGA | | |
| | TGCCAACAGTATCCGTCCGTGACGGTACCGT | | |
| | AAGAGAAAGCCACGGCTAACTACGTGCCAG | | |
| | CAGCC | | |
| BVAB2 | GCGGCTAGATAAGTGTGATGTTTAAATCCAA | 100 | 28 |
| | GGCTTAACCTTGGGGTTCATTACAAACTGTT | | |
| | TAGCTTGAGTGCTGGAGAGGATAGTGGAAT | | |
| | TCCTAGTG | | |
| Candida | GCTTTAGCAGCAATAATGTGTTCAACTAAAG | 121 | 29 |
| albicans | ATCTCTTGTGTTTGAAGGTGTTACCTTTAGC | | |
| | AGCTTTGTACAAGTIGTGGTACAAGTGTTTG | | |
| | TCAATTTTACCAGCATCTCTGTATTTAG | | |
| Candida | TTGGCATAAAGGACCCGTAGGCTTAAGTATA | 123 | 30 |
| albicans | TACATTATATATAATGTGTATTTAATAAGAT | | |
| | AGCCCGTCCTCCAGAGGAGGCCGGGCCATC | | |
| | CTCGATAGAATCTATTAGCTGGTAGTATGTA | | |
| Candida | GCTTTAGCAGCAATAATGTGTTCAACTAAAG | 121 | 31 |
| dubliniensis | ATCTCTTGTGTTTAAAAGTGTTACCTTTGGC | | |
| | AGCTTTGTACAAGTIGTGGTACAAGTGTTTA | | |
| | TCAATTTTACCAGCATCTCTGTATTTAG | | |
| Candida | GCTTTAGCAGCAATAATGTGTTCAACTAAAG | 121 | 32 |
| tropicalis | ATCTCTTGTGTTTGAAAGTGTTACCTTTAGC | | |
| | AGCTTTGTACAAGTIGTGGTATAAGTGTTTG | | |
| | TCAATTTTACCAGCATCTCTGTATTTAG | | |
| Candida | GCCTTAGCAGTGATGATGTGTTCAACCAAGG | 121 | 33 |
| parapsilosis | ATCTCTTGTGTTTAAAGGTGTTACCTTTAGC | | |
| | AGCCTTGTACAAGTIGTGGTACAAGTGTCTG | | |
| | TCAATCTTACCAGCATCTCTGTATTTAG | | |
| Candida | GAGGCCGACTACACGACGACGATCGACAAG | 139 | 34 |
| glabrata | GGCAACGGTGACTTCGAGACAGACCTTGTTT | | |
| | CGCACATCACGACCAAGGACTCTGACGGCA | | |
| | AGCCTACCACGATCACCACCACCATCCCATT | | |
| | GAAGCCAAGTGATGCCG | | |
| Candida | GGACATTCAGATAATCCAGATTTTTCAAAGA | 106 | 35 |
| krusei | TGAGAAAAATTTCTAAAACATCGAATCCCTT | | |
| | ACCCGTGAAGAAATATAGTGGACAAATATC | | |
| | AGCTAATAAAATGA | | |
| Trichomonas | GTAACAACCTTGGAGTTCTTCTTGAGCTGAA | 146 | 36 |
| vaginalis | CACGGACAGCCTTACGAATAGCAGAGTTTG | | |
| | GCTGCTTAGCTTCGACGGCGACCTTGCCGAC | | |
| | GACAATGCCAGATGCCATTGATGTACCTTCG | | |
| | AGTGGTGTCTTGTAGATTGATGT | | |

The present assay relies on the polymerase chain reaction (PCR), and can be carried out in a substantially automated manner using a commercially available nucleic acid amplification system. As described herein, exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif). In some embodiments, the amplification system may be available at the same location as the individual to be tested, such as a health care provider's office, a clinic, or a community hospital, so processing is not delayed by transporting the sample to another facility. The present assay can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERT® utilizes a self-contained, single use cartridge. Sample extraction, amplification, and detection may all carried out within this self-contained sample cartridge as described herein.

After the sample is added to the cartridge, the sample is contacted with lysis buffer and released nucleic acid (NA) is bound to an NA-binding substrate such as a silica or glass substrate. The sample supernatant is then removed and the NA eluted in an elution buffer such as a Tris/EDTA buffer. The eluate may then be processed in the cartridge to detect target genes as described herein. In some embodiments, the eluate is used to reconstitute at least some of the PCR reagents, which are present in the cartridge as lyophilized particles.

In some embodiments, RT-PCR is used to amplify and analyze the presence of the target genes. In some embodiments, the reverse transcription uses MMLV RT enzyme and an incubation of 5 to 20 minutes at 40° C. to 50° C. In some embodiments, the PCR uses Taq polymerase with hot start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed.

In some embodiments, a double-denature method is used to amplify low copy number targets. A double-denature method comprises, in some embodiments, a first denaturation step followed by addition of primers and/or probes for detecting target genes. All or a substantial portion of the nucleic acid-containing sample (such as a DNA eluate) is then denatured a second time before, in some instances, a portion of the sample is aliquotted for cycling and detection of the target genes. While not intending to be bound by any particular theory, the double-denature protocol may increase the chances that a low copy number target gene (or its complement) will be present in the aliquot selected for cycling and detection because the second denaturation effectively doubles the number of targets (i.e., it separates the target and its complement into two separate templates) before an aliquot is selected for cycling. In some embodiments, the first denaturation step comprises heating to a temperature of 90° C. to 100° C. for a total time of 30 seconds to 5 minutes. In some embodiments, the second denaturation step comprises heating to a temperature of 90° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for an NA-containing sample (such as a DNA eluate) may comprise heating at least one, at least two, at least three, or at least four aliquots of the sample separately (either sequentially or simultaneously) to a temperature of 90° C. to 100° C. for 60 seconds each. As a non-limiting example, a second denaturation step for a NA-containing sample (such as a DNA eluate) containing enzyme, primers, and probes may comprise heating at least one, at least two, at least three, or at least four aliquots of the eluate separately (either sequentially or simultaneously) to a temperature of 90° C. to 100° C. for 5 seconds each. In some embodiments, an aliquot is the entire NA-containing sample (such as a DNA eluate). In some embodiments, an aliquot is less than the entire NA-containing sample (such as a DNA eluate).

The present invention is not limited to particular primer and/or probe sequences. Exemplary amplification primers and detection probes are described herein and shown in Table 3 above.

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described here.

Exemplary Data Analysis

In some embodiments, the presence of bacterial vaginosis, vulvovaginal candidiasis/vulvovaginal candidiasis-associated species/Candida spp., trichomoniasis/trichomoniasis-associated species/Trichomonas vaginalis, and/or antifungal resistant Candida/antifungal resistant Candida-associated species is detected if the Ct value for any one of the target genes is below a certain threshold. In some embodiments, the valid range of Ct values is equal or less than 42 cycles. In some embodiments the valid range of Ct values is about 8 to 41.9 Ct. In the case of Megal-BVAB2, a Ct value up to 42.0 is valid. In some such embodiments, if no amplification above background is observed from the bacterial vaginosis-specific primers after 42 cycles, the sample is considered to be negative for bacterial vaginosis. In some such embodiments, the sample is considered to be negative for bacterial vaginosis only if amplification of the exogenous control (SPC) is above background.

As further described herein, bacterial vaginosis is identified if a level of Atopobium spp. is at least a first threshold in the absence of BVAB2 or Megasphaera-1, or the levels of Atopobium spp. and BVAB2 or Megasphaera-1 are at least a second threshold greater than the first threshold, or the levels of Atopobium spp., BVAB2, and Megasphaera-1 are at least a third threshold greater than the second threshold.

In some examples, the cartridge may utilize two channels (Atop gp and Megal-BVAB2) and a BV rules-based algorithm to detect organisms associated with BV (individual organisms are not reported). The primers and probe in the Atop gp channel amplify and detect Atopobium group (A. vaginae and Atopobium novel species CCUG 55226). The primers and probes in the Megal-BVAB2 channel amplify and detect Megasphaera Type 1 (Megasphaera-1) and Bacterial Vaginosis-Associated Bacterium 2 (BVAB2); the test will not report separate results or separate Ct values for these two organisms. A BV positive result can be reported either when both channels report a cycle threshold (Ct) within their respective valid Ct ranges, or when only Atopobium group channel reports a Ct within its valid Ct range and Megal-BVAB2 Ct is 0 (for the latter scenario, the Atopobium group has an earlier cut-off than that in the presence of Megasphaera-1 and/or BVAB2). For example, bacterial vaginosis can be identified if a Ct range/cut-off for Atopobium spp. in the absence of Megasphaera-1 and BVAB2 is equal to or less than 26 (such as from 8 to 26); or a Ct range/cut-off for Atopobium spp. is equal to or less than 33 (such as from 8 to 33) in the presence of Megasphaera-1 and BVAB2, which Ct range/cut-off is equal to or less than 42 (such as from 8 to 42). In other embodiments, bacterial vaginosis is identified if a level of Atopobium spp. is at least 320,000 CFU/mL in the absence of Megasphaera-1 and BVAB2, or the levels of Atopobium spp. and BVAB2 or Megasphaera-1 are at least 2,750 CFU/mL, at least 50 copies/mL, and at least 390 copies/mL, respectively.

In some embodiments, vulvovaginal candidiasis/vulvovaginal candidiasis-associated species/Candida spp. is identified if levels of Candida albicans are at least a first threshold, the levels of Candida dubliniensis are at least a second threshold higher than the first threshold, the levels of Candida tropicalis are at a third threshold between the first and second, and levels of Candida parapsilosis are at a fourth threshold about the same or greater than the second threshold. The threshold can indicate the amount or level of the target organism present. For example, vulvovaginal candidiasis/vulvovaginal candidiasis-associated species/Candida spp. is identified if a level of Candida albicans is at least 30 CFU/mL, or a level of Candida dubliniensis is at least 1,316 CFU/mL, or a level of Candida tropicalis is at least 750 CFU/mL, and/or a level of Candida parapsilosis is at least 1,339 CFU/mL. In some examples, the cartridge may utilize a single channel (Candida group) to detect vulvovaginal candidiasis/vulvovaginal candidiasis-associated species/Candida spp. Individual organisms associated with vulvovaginal candidiasis may not be reported. For example, two sets of oligos can be used to identify Candida spp, one set for the detection of C. albicans, C. tropicalis, C. parapsilosis, C. dubliniensis that targets a segment within the ribosomal protein L19 gene on C. albicans Chromosome 3; the other set for the detection of C. albicans only, and it targets a region in the C. albicans mitochondrial DNA, Region Ca-19.

In some embodiments, trichomoniasis/trichomoniasis-associated species/Trichomonas vaginalis is identified if the levels of Trichomonas vaginalis is at least 5 cells/mL.

In some embodiments, the method further includes detecting nucleic acid sequences characteristic of antifungal resistant Candida spp./antifungal resistant Candida-associated species in a subject, where the subject is identified as having antifungal resistant Candida spp/antifungal resistant Candida-associated species. if level of Candida glabrata is at least 20 CFU/mL and/or Candida krusei at least 656 CFU/mL. In some examples, the cartridge may utilize a single channel (Candida glab-krus) to detect antifungal resistant Candida/antifungal resistant Candida-associated species (Candida glabrata and/or Candida krusei). One oligo set can be specific for the detection of C. glabrata and the oligos target tandem repeat regions on chromosome L; and the other oligo set can be specific for C. krusei, targeting the coding region of a hypothetical protein, possibly a helicase. The biological sample can be a vaginal swab, a vaginal mucus sample, a vaginal tissue sample, or a vaginal cell sample. It is appreciated that in some embodiments, the cartridge can be configured to identify one of bacterial vaginosis, vulvovaginal candidiasis, or trichomoniasis, or any combination thereof, in accordance with the thresholds and levels described above.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a vaginal sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample or sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence of BV, candidiasis, candidiasis associated spp., Candida spp., antifungal resistant candidiasis, antifungal resistant candidiasis associated spp., Trichomonas vaginalis, or trichomoniasis) for the subject, with or without recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot™ (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more nucleotide analogs (i.e., modified nucleotides) discussed herein.

In some embodiments, a polynucleotide is provided that comprises a region that is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the Atopobium vaginae 16S rRNA gene, Megasphaera-1 16S rRNA gene, BVAB2 16S rRNA gene, Candida albicans/Candida dubliniensis/Candida tropicalis/Candida parapsilosis ribosomal protein L19 gene on C. albicans Chromosome 3, Candida albicans mitochondrial DNA, Region Ca-19, Candida glabrata tandem repeat regions on chromosome L, Candida krusei coding region of a hypothetical protein, possibly a helicase, and Trichomonas vaginalis 40S ribosomal protein S23. In some embodiments, a polynucleotide is provided that comprises a region that is at least 85%, at least 90%, at least 95%, or 100% identical to, or complementary to, a span of 6 to 100, 8 to 100, 8 to 75, 8 to 50, 8 to 40, or 8 to 30 contiguous nucleotides of the Atopobium vaginae 16S rRNA gene, Megasphaera-1 16S rRNA gene, BVAB2 16S rRNA gene, Candida albicans/Candida dubliniensis/Candida tropicalis/Candida parapsilosis ribosomal protein L19 gene on C. albicans Chromosome 3, Candida albicans mitochondrial DNA, Region Ca-19, Candida glabrata tandem repeat regions on chromosome L, Candida krusei coding region of a hypothetical protein, possibly a helicase, and Trichomonas vaginalis 40S ribosomal protein S23. Nonlimiting exemplary polynucleotides are shown in Table 3.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of selectively hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET (fluorescence resonance energy transfer) probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). Thus, in some embodiments, the emission spectrum of the dye should overlap considerably with the absorption spectrum of the quencher. In other embodiments, the dye and quencher are not at the ends of the FRET probe.

In some embodiments, detection of each target (Atopobium vaginae, Megasphaera-1, BVAB2, Candida albicans, Candida dubliniensis, Candida tropicalis, Candida parapsilosis, Candida glabrata, Candida krusei, and Trichomonas vaginalis) can be carried out using a single labeled primer or probe, specific for each target. Different primers and/or probes can have the same label. By using primers or probes labeled with different detectable moieties (e.g., different fluorescent reporter dyes), numerous targets can be detected simultaneously in a single reaction tube. In some instances, as many as 10 different labels (such as 4, 5, 6, 7, 8, 9, or 10 labels) can be used in a single reaction chamber or a plurality of reaction chambers. Each target can be independently monitored in such multiplexing technology. In some instances, detection of a plurality of targets (such as Candida albicans, Candida dubliniensis, Candida tropicalis, and Candida parapsilosis) can be carried out using a single labeled primer or probe. A melt curve may be generated in order to distinguish two or more targets that each use the same label, but may not be necessarily required.

Exemplary Polynucleotide Modifications

In some embodiments, the methods of detecting at least one target gene described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications. In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine. In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

Exemplary Primers

In some embodiments, a primer and primer pairs are provided. In some embodiments, a primer is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least at least 21, at least 22, at least 23, at least 24, at least 25, at least 26, at least 27, at least 28, at least 29, or at least 30 contiguous nucleotides of the Atopobium vaginae 16S rRNA gene, Megasphaera-1 16S rRNA gene, BVAB2 16S rRNA gene, Candida albicans/Candida dubliniensis/Candida tropicalis/Candida parapsilosis ribosomal protein L19 gene on C. albicans Chromosome 3, Candida albicans mitochondrial DNA, Region Ca-19, Candida glabrata tandem repeat regions on chromosome L, Candida krusei coding region of a hypothetical protein, possibly a helicase, and/or Trichomonas vaginalis 40S ribosomal protein S23. Nonlimiting exemplary primers are shown in Table A.

In some embodiments, a primer may also comprise portions or regions that are not identical or complementary to the target gene. In some embodiments, a region of a primer that is at least 85%, at least 90%, at least 95%, or 100% identical or complementary to a target gene is contiguous, such that any region of a primer that is not identical or complementary to the target gene does not disrupt the identical or complementary region.

In some embodiments, a primer comprises a portion that is at least 85%, at least 90%, at least 95%, or 100% identical to a region of a target gene. In some such embodiments, a primer that comprises a region that is at least 85%, at least 90%, at least 95%, or 100% identical to a region of the target gene is capable of selectively hybridizing to a cDNA that has been reverse transcribed from the RNA, or to an amplicon that has been produced by amplification of the target gene. In some embodiments, the primer is complementary to a sufficient portion of the cDNA or amplicon such that it selectively hybridizes to the cDNA or amplicon under the conditions of the particular assay being used. As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. Exemplary hybridization conditions are discussed herein, for example, in the context of a reverse transcription reaction or a PCR amplification reaction. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein.

In some embodiments, a primer comprises a detectable moiety.

In some embodiments, two forward primers or two reverse primers may be used to amplify a target gene, resulting in two different amplicons. In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long. Nonlimiting exemplary primer pairs are shown in Table 3.

### VI. Experimental Results

### A. Clinical Results and Performance Testing

To evaluate the clinical performance of the MVP cartridge test, a blinded clinical study was conducted at 15 geographically diverse sites in the U.S. Subjects included female patients≥14 years of age who presented with signs and/or symptoms of vaginosis/vaginitis. For eligible subjects, one (1) self-collected swab (SVS) sample collected in a clinical setting and five (5) clinician-collected vaginal swab (CVS) specimens were obtained for testing with the MVP cartridge test and reference/comparator testing. Patient management continued at the site per the standard practice, independent of investigational test results.

The MVP cartridge test performance was compared to the following reference/comparator methods: a FDA-cleared nucleic acid amplification test (NAAT) for the BV target, yeast culture followed by mass spectrometry identification for the Candida group and Candida glab-krus targets, a patient infected status (PIS) algorithm that included a combination of NAAT and culture results for the TV target. When applicable, investigation of discrepant results was performed by testing specimens with another FDA-cleared NAAT. The study population comprised of 1,407 female patients 14 to ≥50 years of age. A total of 2,805 vaginal swabs were tested and were eligible for inclusion in the MVP study.

Overall performance of the MVP test is presented in Table 5. The MVP test demonstrated positive percent agreement (PPA) and negative percent agreement (NPA) of 93.8% and 93.8% for BV detection in CVS specimens, respectively, and 94% and 92.9% in SVS specimens, respectively. For Candida group detection, the MVP test demonstrated sensitivity and specificity of 98% and 94.6% in CVS specimens, respectively, and 97.5% and 92.1% in SVS specimens, respectively. The MVP test demonstrated sensitivity and specificity of 93.6% and 99.6% for Candida glab-krus detection in CVS fresh specimens, respectively, and 97.8% and 99.4% in SVS fresh specimens, respectively. For TV detection, the MVP test demonstrated PPA and NPA of 97.3% and 99.6% in CVS specimens, respectively, and 97.3% and 99.8% in SVS specimens, respectively.

TABLE 5
Overall Performance of the MVP Test

| | Clinician-collected (CVS) | | Self-collected (SVS) | |
|---|---|---|---|---|
| | Sensitivity/PPA | Specificity/NPA | Sensitivity/PPA | Specificity/NPA |
| | (95% CI) | (95% CI) | (95% CI) | (95% CI) |
| BV | 93.8% | 93.8% | 94.0% | 92.9% |
| | 531/566 | 808/861 | 533/567 | 794/855 |
| | (91.5%-95.6%) | (92.0%-95.3%) | (91.7%-95.7%) | (90.9%-94.4%) |
| Candida group* | 98.0% | 94.6% | 97.5% | 92.1% |
| | 396/404 | 984/1040 | 393/403 | 954/1036 |
| | (96.1%-99.0%) | (93.1%-95.8%) | (95.5%-98.7%) | (90.3%-93.6%) |
| Candida | 93.6% | 99.6% | 97.8% | 99.4% |
| glab-krus | 44/47 | 1392/1397 | 45/46 | 1384/1393 |
| Fresh Prospective | (82.8%-97.8%) | (99.2%-99.9%) | (88.7%-99.6%) | (98.8%-99.7%) |
| Candida | 99.0% | 96.4% | N/A | N/A |
| glab-krus | 98/99 | 27/28 | | |
| Contrived** | (94.5%-99.8%) | (82.3%-99.4%) | | |
| TV | 97.3% | 99.6% | 97.3% | 99.8% |
| Overall | 73/75 | 1332/1337 | 72/74 | 1330/1333 |
| | (90.8%-99.3%) | (99.1%-99.8%) | (90.7%-99.3%) | (99.3%-99.9%) |

*Target includes C. albicans, C. tropicalis, C. parapsilosis, and C. dubliniensis
**Contrived specimens were prepared using individual negative clinical CVS and SVS specimens.

### B. Multi-Target Detection Results

The number of fresh specimens with positive results for more than one target as determined by the MVP test or reference/comparator methods are summarized in Table 6, where bolded values indicate concordant results and non-bolded values indicate discordant results.

Among 1,433 CVS specimens, 191 specimens yielded multi-target concordant results between MVP test and reference methods. Of the 191 specimens, 66% (126/191) had concordant BV and Candida group co-infections, and 23.6% (45/191) had concordant BV and TV co-infections. Among 1,428 SVS specimens, 183 specimens yielded multi-target concordant results. Of the 183 specimens, 65% (119/183) had concordant BV and Candida group co-infections, and 24% (44/183) had concordant BV and TV co-infections.

TABLE 6
Multi-Target Detection by the MVP Test
Total Number of Occurrences between the Xpert Xpress MVP Test vs. Reference/Comparator Method (CVS/SVS)

| | | | | | BV, | | | | | | | | | |
|---|---|---|---|---|---|---|---|---|---|---|---|---|---|---|
| | | | | | Candida | | BV, | | Candida | | | | | |
| | | | BV, | BV, | group, | | Candida | | group, | Candida | | Candida | | |
| | | | Candida | Candida | Candida | BV, | group, | Candida | Candida | group, | Candida | glab-krus, | | |
| | Infections | BV | group | glab-krus | glab-krus | TV | TV | group | glab-krus | TV | glab-krus | TV | TV | Negative |
| T [symbol] | BV | | 0/5 | — | — | — | — | 1/0 | — | — | — | — | — | 26/28 |
| | BV, | 16/26 | 126/119 | — | 1/0 | — | — | 16/22 | 1/0 | — | — | — | — | 1/2 |
| | Candida | | | | | | | | | | | | | |
| | group | | | | | | | | | | | | | |
| | BV, | 2/3 | — | 3/1 | — | — | — | — | — | — | 2/3 | — | — | — |
| | Candida | | | | | | | | | | | | | |
| | glab- | | | | | | | | | | | | | |
| | krus | | | | | | | | | | | | | |
| | BV, | 0/1 | — | 0/2 | 4/5 | — | — | — | 0/1 | — | 1/1 | — | — | — |
| | Candida | | | | | | | | | | | | | |
| | group, | | | | | | | | | | | | | |
| | Candida | | | | | | | | | | | | | |
| | glab- | | | | | | | | | | | | | |
| | krus | | | | | | | | | | | | | |
| | BV, | 2/2 | — | — | — | 45/44 | 1/1 | — | — | — | — | — | 4/5 | — |
| | TV | | | | | | | | | | | | | |
| | BV, | — | — | — | — | 3/3 | 9/9 | — | — | 1/0 | — | — | — | — |
| | Candida | | | | | | | | | | | | | |
| | group, | | | | | | | | | | | | | |
| | TV | | | | | | | | | | | | | |
| | Candida | 1/2 | 14/15 | — | — | — | — | | — | 1/1 | 1/1 | — | — | 27/36 |
| | group | | | | | | | | | | | | | |
| | Candida | — | — | 1/1 | — | — | — | — | 1/1 | — | 3/6 | — | — | — |
| | group, | | | | | | | | | | | | | |
| | Candida | | | | | | | | | | | | | |
| | glab- | | | | | | | | | | | | | |
| | krus | | | | | | | | | | | | | |
| | Candida | — | — | — | — | — | — | 1/0 | — | 2/3 | — | — | — | 1/1 |
| | group, | | | | | | | | | | | | | |
| | TV | | | | | | | | | | | | | |
| | Candida | — | — | — | — | — | — | — | — | — | 1/0 | — | — | — |
| | group, | | | | | | | | | | | | | |
| | Candida | | | | | | | | | | | | | |
| | glab- | | | | | | | | | | | | | |
| | krus, | | | | | | | | | | | | | |
| | TV | | | | | | | | | | | | | |
| | Candida | — | — | 1/0 | — | — | — | — | — | — | | — | — | 3/5 |
| | glab- | | | | | | | | | | | | | |
| | krus | | | | | | | | | | | | | |
| | Candida | — | — | — | — | — | — | — | — | — | — | 1/1 | — | — |
| | glab- | | | | | | | | | | | | | |
| | krus, | | | | | | | | | | | | | |
| | TV | | | | | | | | | | | | | |
| | TV | — | — | — | — | 1/1 | — | — | — | — | — | — | | — |
| | Negative | 17/15 | — | — | — | — | — | 6/4 | — | — | — | — | 1/1 | |

[symbol]

indicates data missing or illegible when filed

Of the 2,947 MVP runs performed in the clinical study, 130 resulted in non-determinate (“Error”, “Invalid” or “No Results”) results on first attempt. Upon retest of these 130 specimens, 22 remained non-determinate. The initial non-determinate rate was 4.4% (130/2947) and the overall non-determinate rate was 0.7% (22/2947). The initial non-determinate rate for CVS specimens was 3.9% (58/1473) and the overall non-determinate rate was 0.5% (8/1473). The initial non-determinate rate for SVS specimens was 4.9% (72/1474) and the overall non-determinate rate was 0.9% (14/1474).

1. Analytical Sensitivity (Limit of Detection)

The analytical sensitivity of the MVP test was determined by preparing dilutions for each of the target organisms detected by the test. The near cut-off concentrations for the BV organisms were also determined. Positive samples were prepared by inoculating simulated vaginal swab matrix with each representative strain or quantified stocks of plasmid DNA containing the cloned genomic targets of BVAB2 or Megasphaera-1. Replicates of 20 were evaluated at a minimum of five concentrations for each of the target organisms. The limit of detection (LoD) and near cut-off concentrations for the target organisms were estimated by probit analysis. The LoD is defined as the lowest concentration of organism sample that can be reproducibly distinguished from negative samples with 95% confidence. The near cut-off concentration for the BV organisms is defined as the lowest concentrations of Atopobium vaginae and Megasphaera-1, or A. vaginae and BVAB2, or A. vaginae and Megasphaera-1 and BVAB2, or A. vaginae in the absence of Megasphaera-1 and BVAB2 that result in BV POSITIVE test results and can be reproducibly distinguished from negative samples with a 95% confidence level. The LoD for each Candida spp. and Trichomonas vaginalis strain was confirmed in natural clinical vaginal swab matrix and simulated vaginal swab matrix (Table 7). The LoD and near cut-off concentrations for each BV organism were confirmed in a simulated vaginal swab matrix (Tables 7 and 8).

TABLE 7
Limit of Detection of BV, Candida group, Candida
glab-krus, and TV Targets for Xpert Xpress MVP

| Target | Strain | LoD | Units | Ct range |
|---|---|---|---|---|
| BV | Atopobium vaginae ATCC BAA- | 32 | CFU/mL | |
| | 55 | | | |
| | Megasphaera-1 plasmid DNA | 338 | copies/mL | |
| | BVAB2 plasmid DNA | 50 | copies/mL | |
| Candida group | Candida albicans ATCC 32032 | 30 | CFU/mL | 8-40 |
| | Candida dubliniensis ATCC | 1,316 | CFU/mL | 8-40 |
| | 44508 | | | |
| | Candida tropicalis ATCC 13803 | 750 | CFU/mL | 8-40 |
| | Candida parapsilosis ATCC | 1,339 | CFU/mL | 8-40 |
| | 22019 | | | |
| Candida glab- | Candida glabrata ATCC 28482 | 20 | CFU/mL | 8-40 |
| krus | Candida krusei ATCC 34135 | 656 | CFU/mL | 8-40 |
| TV | Trichomonas vaginalis ATCC | 5 | cells/mL | 13-45 |
| | 30001 | | | |

TABLE 8
Near Cut-off Concentration of BV Target for Xpert Xpress MVP

| | | Near Cut-off | | Valid |
|---|---|---|---|---|
| Target | Strain | concentration | Units | max Ct |
| BV | Atopobium vaginae ATCC BAA-55 | 320,000 | CFU/mL | 26 |
| | (in the absence of Megasphaera-1 and | | | |
| | BVAB2) | | | |
| | Atopobium vaginae ATCC BAA-55 | 2,750 | CFU/mL | 33 |
| | (in the presence of Megasphaera-1 | | | |
| | and/or BVAB2) | | | |
| | Megasphaera-1 plasmid DNA | 390 | copies/mL | 42 |
| | BVAB2 plasmid DNA | 50 | copies/mL | 42 |

2. Analytical Reactivity (Inclusivity)

The analytical reactivity (inclusivity) of the MVP test was determined with 5 strains of Candida albicans, 5 strains of C. dubliniensis, 5 strains of C. tropicalis, 5 strains of C. parapsilosis, 5 strains of C. glabrata, 5 strains of C. krusei, 11 strains of Atopobium spp. (Atopobium vaginae and/or Atopobium novel species CCUG 55226), and 10 strains of Trichomonas vaginalis that were diluted in simulated vaginal swab matrix at 3×LoD. Each Atopobium spp. strain was also evaluated at 3× near cut-off concentrations diluted in simulated vaginal swab matrix in the absence or presence of BVAB2 and/or Megasphaera-1 DNA to confirm the correct BV POSITIVE test results were reported. Three replicates were tested for each strain.

The MVP test correctly identified 46 of 51 strains upon initial testing at 3×LoD. Two strains of Atopobium vaginae tested at 3×LoD and three strains of Candida albicans tested at 3×LoD were not detected and were tested at higher concentrations to determine the minimum concentration sufficient for detection. One A. vaginae strain was detected at ˜4×LoD and the other strain was detected at −12×LoD. One C. albicans strain was detected at −4×LoD and the other two C. albicans strains were detected at −20×LoD. For near cut-off concentration of Atopobium spp. in the absence of Megasphaera-1 and BVAB2, the MVP test correctly reported BV POSITIVE test result for 7 of the 11 strains upon initial testing at 3× near cut-off concentration. Four strains did not meet acceptance criteria and were further tested to determine the minimum concentration sufficient for reporting BV POSITIVE test result. One Atopobium spp. strain reported BV POSITIVE at ˜4×, two strains at ˜6×, and one strain at ˜12× near cut-off concentration. For the near cut-off concentration of Atopobium spp. in the presence of Megasphaera-1 and/or BVAB2, the MVP test correctly reported BV POSITIVE test result for 7 of the 11 strains tested upon initial testing at 3× near cut-off concentration. Four strains did not meet acceptance criteria and were further tested to determine the minimum concentration sufficient for reporting BV POSITIVE test result. Two Atopobium spp. strains reported BV POSITIVE at ˜4×, one strain at ˜6×, and one strain at ˜7× near cut-off concentration.

3. Analytical Specificity (Cross-reactivity)

The analytical specificity of the MVP test was evaluated by testing a panel of 115 potentially cross-reactive microorganisms that are likely to be found in the vaginal flora/female genital tract. All strains were tested in triplicates in simulated vaginal swab matrix at a concentration of at least 10⁶ CFU/mL, 10⁵ cells/mL, 10⁵ TCID₅₀/mL, or 10⁴ International Unit (IU)/mL. No cross-reactivity was observed for 112 of the 115 microorganisms tested with the MVP test at likely found concentrations. Trichomonas tenax and Pentatrichomonas hominis tested at 1×10⁵ cells/mL reported TV DETECTED with the MVP test. Candida orthopsilosis tested at 1×10⁶ CFU/mL reported Candida group DETECTED with the MVP test. All three initially cross-reactive organisms were negative on retest at lower concentrations.

4. Microbial Interference

An interfering microorganism study was performed to assess the inhibitory effects of microorganisms that may be encountered in vaginal specimens on the performance of the MVP test. Thirteen microorganisms were tested for potential interference at ≥10⁶ CFU/mL for bacteria and at ≥10⁴ International Unit/mL or cells/mL for viruses. Each of the microorganisms was tested in simulated vaginal swab matrix in the presence and absence of Atopobium vaginae at 3× near cut-off concentrations, Megasphaera-1 and BVAB2 targets each at ˜1.5× near cut-off concentrations, and Candida albicans, C. glabrata and Trichomonas vaginalis targets each at 3×LoD. The results showed that the presence of the tested microorganisms did not interfere with the performance of the MVP test.

5. Competitive Interference

Competitive interference between targets (BV, Candida group, Candida glab-krus and TV) of the MVP test caused by co-infections was evaluated by testing each target at low positive concentration in the presence of another target at high concentration in simulated vaginal swab matrix. Competitive inhibitory effects between the BV analytes (Atop gp and Megal-BVAB2) were also evaluated in simulated vaginal swab matrix. Under the conditions of this study, competitive inhibitory effects were not observed between MVP targets or BV analytes with the MVP test.

6. Carry-over Contamination

A study was conducted to demonstrate that single-use, self-contained GeneXpert cartridges prevent specimen and amplicon carry-over contamination from very high titer positive samples into successively run negative samples when processed in the same GeneXpert module. The study consisted of a negative sample processed in the same GeneXpert module immediately after processing a very high BV positive sample (an A. vaginae strain at 2.8×10⁷ CFU/mL and BVAB2 plasmid DNA at 5.0×10⁸ copies/mL), a very high Candida group sample (a C. albicans strain at 3.0×10⁶ CFU/mL), or a very high TV sample (a T vaginalis strain at 5.0×10⁶ cells/mL) in simulated vaginal swab matrix. The testing scheme was repeated 20 times in a single GeneXpert module for a total of 41 runs (20 high positive samples and 21 negative samples per module) across 3 GeneXpert modules. There was no evidence of any carry-over contamination. All 63 negative samples were correctly reported as negative/not detected. All 60 positive samples were correctly reported as positive/detected.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art. Unless stated otherwise, the term “about” is considered to mean within +/−10%. It is further appreciated that the various listings and groups of species can be incorporated into an open group (e.g. “comprising”) of species or within a closed group (e.g. “consisting”) of species in the recited combination or any combination thereof. Any references to publication, patents, or patent applications are incorporated herein by reference in their entirety for all purposes.

What is claimed is:

1. A method for detecting and/or identifying bacterial vaginosis (BV), Candida spp., Trichomonas vaginalis, or a combination thereof in a biological sample obtained from a subject, the method comprising:
placing the biological sample in a cartridge comprising a cartridge body having a plurality of chambers in fluidic communication, a reaction vessel configured for amplification of the nucleic acid by thermal cycling and comprising at least one amplification region, a fluidic path between the plurality of chambers and the reaction vessel, and a filter in the fluidic path;
lysing cells in the biological sample with one or more lysis reagents present within at least one of the plurality of chambers and capturing deoxyribonucleic acid (DNA) released therefrom;
subjecting the DNA, primers, and probes to amplification conditions;
detecting the level of Atopobium spp., BVAB2, Megasphaera-1, Candida spp., and Trichomonas vaginalis in the biological sample, wherein the primers and probes are present within at least one amplification region of the reaction vessel; and
identifying the presence of bacterial vaginosis, Candida spp., Trichomonas vaginalis, or a combination thereof in the subject, or determining that bacterial vaginosis, Candida spp., or Trichomonas vaginalis is not present, based on the detected levels of each of: Atopobium spp., BVAB2, Megasphaera-1, Candida spp., and Trichomonas vaginalis.

2. The method of claim 1, wherein bacterial vaginosis is identified if
(i) the detected level of Atopobium spp. is at least 320,000 CFU/mL in the absence of BVAB2 and Mega-1, or
(ii) the detected levels of Atopobium spp. is at least 2,750 CFU/mL and the level of BVAB2 is at least 50 copies/mL and/or the level of Megasphaera-1 is at least 390 copies/mL.

3. The method of claim 1, wherein the method further comprises amplifying and detecting nucleic acid sequences characteristic of antifungal resistant Candida spp. comprising Candida glabrata and Candida krusei in a subject, wherein the subject is identified as having a second group of Candida spp. if the level of Candida glabrata is at least 20 CFU/mL or the level of Candida krusei is at least 656 CFU/mL.

4. The method of claim 1, wherein the biological sample is a vaginal swab, a vaginal mucus sample, a vaginal tissue sample, or a vaginal cell sample.

5. The method of claim 1, wherein the identifying is done at the same facility where the biological sample was collected from the subject.

6. The method of claim 1, wherein the method is a Clinical Laboratory Improvement Amendments (CLIA)-waived test.

7. The method of claim 1, wherein the presence or absence of bacterial vaginosis, Candida spp., Trichomonas vaginalis, or a combination thereof is detected within the biological sample within minutes or within 60 minutes of collecting the sample from the subject.

8. The method of claim 1, wherein subjecting the DNA, primers, and probes to amplification conditions to detect the level of Atopobium spp., BVAB2, Megasphaera-1, Candida spp., and Trichomonas vaginalis is carried out in a single amplification region of the reaction vessel.

9. The method of claim 1, wherein
the primers and probes for amplifying and detecting Atopobium spp. are capable of hybridizing to the 16S rRNA gene of Atopobium vaginae;
the primers and probes for amplifying and detecting BVAB2 are capable of hybridizing to the 16S rRNA gene of BVAB2;
the primers and probes for amplifying and detecting Megasphaera-1 are capable of hybridizing to the 16S rRNA gene of Megasphaera-1;
the primers and probes for amplifying and detecting Candida spp. are capable of hybridizing to the ribosomal protein L19 gene of Candida albicans, the region Ca-19 (Mitochondrial DNA) gene of Candida albicans, tandem repeat regions on chromosome L gene of Candida glabrata, and a coding region of a hypothetical protein of Candida krusei; and
the primers and probes for amplifying and detecting the presence of Trichomonas vaginalis are capable of hybridizing to the 40S ribosomal protein S23 gene of Trichomonas vaginalis.

10. The method of claim 1, wherein the
the primers and probes for amplifying and detecting the presence of Atopobium vaginae comprise a sequence that is identical or complementary to at least 15 contiguous nucleotides of one or more of SEQ ID NO: 1, 2, and 3;
the primers and probes for amplifying and detecting the presence of BVAB2 comprise a sequence that is identical or complementary to at least 15 contiguous nucleotides of one or more of SEQ ID NO: 4, 5, and 6;
the primers and probes for amplifying and detecting the presence of Megasphaera-1 comprise a sequence that is identical or complementary to at least 15 contiguous nucleotides of one or more of SEQ ID NO: 7, 8, and 9;
the primers and probes for amplifying and detecting the presence of Candida spp. comprise a sequence that is identical or complementary to at least 15 contiguous nucleotides of one or more of SEQ ID NO: 10, 11, 12, 13, 14, 15, and 16;
the primers and probes for amplifying and detecting the presence of Candida glabrata comprise a sequence that is identical or complementary to at least 15 contiguous nucleotides of one or more of SEQ ID NO: 17, 18, and 19;
the primers and probes for amplifying and detecting the presence of Candida krusei comprise a sequence that is identical or complementary to at least 15 contiguous nucleotides of one or more of SEQ ID NO: 20, 21, and 22; and
the primers and probes for amplifying and detecting the presence of Trichomonas vaginalis comprise a sequence that is identical or complementary to at least 15 contiguous nucleotides of one or more of SEQ ID NO: 23, 24, and 25.

11. The method of claim 1, wherein the primers and/or probes comprise a detectable label.

12. The method of claim 1, wherein the method for detecting and/or identifying bacterial vaginosis (BV) do not comprise detection of Lactobacillus bacteria or Gardnerella vaginalis.

13. The method of claim 1, further comprising administering a treatment regimen to the subject based on the assessed diagnosis.

14. A cartridge for performing a multiplex vaginal panel (MVP) for identifying bacterial vaginosis, Candida spp., Trichomonas vaginalis, or a combination thereof in a subject, comprising:
a cartridge body having a plurality of chambers defined therein, wherein the plurality of chambers are in in fluidic communication through a fluidic path of the cartridge,
a reaction vessel configured for amplification of the nucleic acid by thermal cycling and comprising a reaction chamber, wherein the reaction vessel comprises at least one amplification region and is attached to the cartridge body and fluidically coupled to the fluidic path of the cartridge; and
a filter disposed in the fluidic path between the plurality of chambers and the reaction vessel;
wherein the plurality of chambers and/or the reaction chamber independently comprise lysis reagents for releasing nucleic acid from a sample, and primers and probes for amplification and detection of DNA sequences characteristic of BVAB2, Megasphaera-1, Atopobium spp., Candida spp., and Trichomonas vaginalis.

15. The cartridge of claim 14, wherein:
the plurality of chambers include:
a sample chamber having at least a fluid outlet in fluid communication with another chamber of the plurality; and
a lysis chamber in fluidic communication with the sample chamber, the lysis chamber comprising lysis reagents for releasing nucleic acid, optionally wherein the sample chamber and lysis chamber are the same;
and
the at least one amplification region comprises a plurality of primers and/or probes disposed therein for the amplification and detection of DNA sequences characteristics of BVAB2, Megasphaera-1, Atopobium spp., Candida spp., and Trichomonas vaginalis.

16. The cartridge of claim 14, wherein the cartridge body further comprises a valve assembly configured to rotate and having at least two ports to fluidically connect the chambers.

17. The cartridge of claim 16, wherein the valve assembly is resistant to alkali corrosion.

18. The cartridge of claim 16, wherein the valve assembly is derived from a polycarbonate material annealed at a temperature of at least 100° C. for at least 1 hour.

19. The cartridge of claim 14, wherein the lysis reagents comprise a guanidinium compound, sodium hydroxide, EDTA, a buffer, and a detergent.

20. The cartridge of claim 19, wherein the sodium hydroxide is isolated from the lysis reagents in one of the plurality of chambers prior to lysing the sample.

21. The cartridge of claim 14, wherein the reaction vessel comprises one amplification region for analyzing a nucleic acid.

22. The cartridge of claim 14, wherein the reaction vessel comprises at least 2 independent amplification regions.

23. The cartridge of claim 14, wherein at least one of the plurality of chambers comprises lyophilized reagents, preferably wherein the lyophilized reagents is in the form of one or more beads.

24. The cartridge of claim 14, wherein
the primers and probes for amplifying and detecting Atopobium spp. are capable of hybridizing to the 16S rRNA gene of Atopobium vaginae;
the primers and probes for amplifying and detecting BVAB2 are capable of hybridizing to the 16S rRNA gene of BVAB2;
the primers and probes for amplifying and detecting Megasphaera-1 are capable of hybridizing to the 16S rRNA gene of Megasphaera-1;
the primers and probes for amplifying and detecting Candida spp. are capable of hybridizing to the ribosomal protein L19 gene of Candida albicans, the region Ca-19 (Mitochondrial DNA) gene of Candida albicans, tandem repeat regions on chromosome L gene of Candida glabrata, and a coding region of a hypothetical protein of Candida krusei; and
the primers and probes for amplifying and detecting the presence of Trichomonas vaginalis are capable of hybridizing to the 40S ribosomal protein S23 gene of Trichomonas vaginalis.

25. The cartridge of claim 14, wherein the cartridge further comprises primers and probes to amplify and detect nucleic acid sequences characteristic of antifungal resistant Candida spp. comprising Candida glabrata and Candida krusei in a subject.

26. The cartridge of claim 14, wherein each component of the cartridge is stable from 2-30° C. for at least 5 months, preferably each component of the cartridge is stable at ambient temperature (about 25° C.) for at least 9 months, more preferably each component of the cartridge is stable at ambient temperature (about 25° C.) for at least 12 months.

27. A system for performing a multiplex vaginal panel (MVP) for identifying bacterial vaginosis, Candida spp., Trichomonas vaginalis, or a combination thereof in a subject, the system comprising:
a module having a receiving bay for receiving a cartridge, wherein the module includes one or more mechansims within the receiving bay for manipulating a fluid sample within the cartridge, and an instrument that interfaces with the reaction vessel;
a memory having programmable instructions recorded thereon, that are specially configured to operate the module according to an MVP assay panel protocol to detect DNA sequence characteristics of BVAB2, Megasphaera-1, Atopobium spp., Candida spp., and Trichomonas vaginalis.

28. The system of claim 27, wherein the instructions are further configured to:
identify bacterial vaginosis if
(i) the detected level of Atopobium spp. is at least a first threshold, or
(ii) the detected level of Atopobium spp. is at least a second threshold that is lower than the first threshold and the detected level of BVAB2 or Megasphaera-1 is at least a third threshold, or
(iii) the detected level of Atopobium spp., is at least a fourth threshold that is lower than the second threshold and both BVAB2 and Megasphaera-1 are detected at levels that are at least a fifth threshold.


SEQUENCE LISTING (36 sequences)

SEQ ID NO: 1 - DNA, 23 bp, synthetic construct
  misc_feature 1..23: note = Description of sequence: Primer; target: Atopobium vaginae; use: Forward
  source 1..23: mol_type = other DNA
cttcggatgtgtataaagtggcg

SEQ ID NO: 2 - DNA, 24 bp, synthetic construct
  misc_feature 1..24: note = Description of sequence: Primer; target: Atopobium vaginae; use: Reverse
  source 1..24: mol_type = other DNA
aggttaataccggatactccatat

SEQ ID NO: 3 - DNA, 23 bp, synthetic construct
  misc_feature 1..23: note = Description of sequence: Primer; target: Atopobium vaginae; use: Probe
  source 1..23: mol_type = other DNA
tttgcactgggatagcctcggga

SEQ ID NO: 4 - DNA, 24 bp, synthetic construct
  misc_feature 1..24: note = Description of sequence: Primer; target: Megasphaera-1; use: Forward
  source 1..24: mol_type = other DNA
gcgtgagtgaagaaggtcttcgga

SEQ ID NO: 5 - DNA, 29 bp, synthetic construct
  misc_feature 1..29: note = Description of sequence: Primer; target: Megasphaera-1; use: Reverse
  source 1..29: mol_type = other DNA
cgaaaaagacggatgccaacagtatccgt

SEQ ID NO: 6 - DNA, 21 bp, synthetic construct
  misc_feature 1..21: note = Description of sequence: Primer; target: Megasphaera-1; use: Probe
  source 1..21: mol_type = other DNA
gctaactacgtgccagcagcc

SEQ ID NO: 7 - DNA, 23 bp, synthetic construct
  misc_feature 1..23: note = Description of sequence: Primer; target: BVAB2; use: Forward
  source 1..23: mol_type = other DNA
gcggctagataagtgtgatgttt

SEQ ID NO: 8 - DNA, 28 bp, synthetic construct
  misc_feature 1..28: note = Description of sequence: Primer; target: BVAB2; use: Reverse
  source 1..28: mol_type = other DNA
gctggagaggatagtggaattcctagtg

SEQ ID NO: 9 - DNA, 29 bp, synthetic construct
  misc_feature 1..29: note = Description of sequence: Primer; target: BVAB2; use: Probe
  source 1..29: mol_type = other DNA
ccttggggttcattacaaactgtttagct

SEQ ID NO: 10 - DNA, 26 bp, synthetic construct
  misc_feature 1..26: note = Description of sequence: Primer; target: Candida albicans/ dubliniensis/ tropicalis/ parapsilosis; use: Forward
  source 1..26: mol_type = other DNA
gctttagcagcaataatgtgttcaac

SEQ ID NO: 11 - DNA, 28 bp, synthetic construct
  misc_feature 1..28: note = Description of sequence: Primer; target: Candida albicans/ dubliniensis/ tropicalis/ parapsilosis; use:
  source 1..28: mol_type = other DNA
gccttagcagtgatgatgtgttcaacca

SEQ ID NO: 12 - DNA, 25 bp, synthetic construct
  misc_feature 1..25: note = Description of sequence: Primer; target: Candida albicans/ dubliniensis/ tropicalis/ parapsilosis; use: Reverse
  source 1..25: mol_type = other DNA
attttaccagcatctctgtatttag

SEQ ID NO: 13 - DNA, 28 bp, synthetic construct
  misc_feature 1..28: note = Description of sequence: Primer; target: Candida albicans/ dubliniensis/ tropicalis/ parapsilosis; use: Probe
  source 1..28: mol_type = other DNA
tgttacctttagcagctttgtacaagtt

SEQ ID NO: 14 - DNA, 21 bp, synthetic construct
  misc_feature 1..21: note = Description of sequence: Primer; target: Candida albicans; use: Forward
  source 1..21: mol_type = other DNA
ttggcataaaggacccgtagg

SEQ ID NO: 15 - DNA, 29 bp, synthetic construct
  misc_feature 1..29: note = Description of sequence: Primer; target: Candida albicans; use: Reverse
  source 1..29: mol_type = other DNA
cgatagaatctattagctggtagtatgta

SEQ ID NO: 16 - DNA, 33 bp, synthetic construct
  misc_feature 1..33: note = Description of sequence: Primer; target: Candida albicans; use: Probe
  source 1..33: mol_type = other DNA
tgtgtatttaataagatagcccgtcctccagag

SEQ ID NO: 17 - DNA, 23 bp, synthetic construct
  misc_feature 1..23: note = Description of sequence: Primer; target: Candida glabrata; use: Forward
  source 1..23: mol_type = other DNA
gaggccgactacacgacgacgat

SEQ ID NO: 18 - DNA, 25 bp, synthetic construct
  misc_feature 1..25: note = Description of sequence: Primer; target: Candida glabrata; use: Reverse
  source 1..25: mol_type = other DNA
atcccattgaagccaagtgatgccg

SEQ ID NO: 19 - DNA, 22 bp, synthetic construct
  misc_feature 1..22: note = Description of sequence: Primer; target: Candida glabrata; use: Probe
  source 1..22: mol_type = other DNA
aaggactctgacggcaagccta

SEQ ID NO: 20 - DNA, 30 bp, synthetic construct
  misc_feature 1..30: note = Description of sequence: Primer; target: Candida krusei; use: Forward
  source 1..30: mol_type = other DNA
ggacattcagataatccagatttttcaaag

SEQ ID NO: 21 - DNA, 26 bp, synthetic construct
  misc_feature 1..26: note = Description of sequence: Primer; target: Candida krusei; use: Reverse
  source 1..26: mol_type = other DNA
tggacaaatatcagctaataaaatga

SEQ ID NO: 22 - DNA, 28 bp, synthetic construct
  misc_feature 1..28: note = Description of sequence: Primer; target: Candida krusei; use: Probe
  source 1..28: mol_type = other DNA
aaacatcgaatcccttacccgtgaagaa

SEQ ID NO: 23 - DNA, 26 bp, synthetic construct
  misc_feature 1..26: note = Description of sequence: Primer; target: Trichomonas vaginalis; use: Forward
  source 1..26: mol_type = other DNA
gtaacaaccttggagttcttcttaag

SEQ ID NO: 24 - DNA, 26 bp, synthetic construct
  misc_feature 1..26: note = Description of sequence: Primer; target: Trichomonas vaginalis; use: Reverse
  source 1..26: mol_type = other DNA
tcaagtggtgtcttgtagattgatgt

SEQ ID NO: 25 - DNA, 22 bp, synthetic construct
  misc_feature 1..22: note = Description of sequence: Primer; target: Trichomonas vaginalis; use: Probe
  source 1..22: mol_type = other DNA
agtttggctgcttagcttcgac

SEQ ID NO: 26 - DNA, 106 bp, Atopobium vaginae
  source 1..106: mol_type = genomic DNA
cttcggatgtgtataaagtggcgaacggctgagtaacacgtgggcaacctgccctttgcactgggatagcctcgggaaaccgaggttaataccggatactccatat

SEQ ID NO: 27 - DNA, 126 bp, unidentified
  source 1..126: mol_type = genomic DNA
  misc_feature 1..126: note = Megasphaera phylotypes 1
gcgtgagtgaagaaggtcttcggattgtaaaactctgttatacgggacgaaaaagacggatgccaacagtatccgtccgtgacggtaccgtaagagaaagccacggctaactacgtgccagcagcc

SEQ ID NO: 28 - DNA, 100 bp, unidentified
  source 1..100: mol_type = genomic DNA
  misc_feature 1..100: note = Bacterial Vaginosis-Associated Bacterium 2
gcggctagataagtgtgatgtttaaatccaaggcttaaccttggggttcattacaaactgtttagcttgagtgctggagaggatagtggaattcctagtg

SEQ ID NO: 29 - DNA, 121 bp, Candida albicans
  source 1..121: mol_type = genomic DNA
gctttagcagcaataatgtgttcaactaaagatctcttgtgtttgaaggtgttacctttagcagctttgtacaagttgtggtacaagtgtttgtcaattttaccagcatctctgtatttag

SEQ ID NO: 30 - DNA, 123 bp, Candida albicans
  source 1..123: mol_type = genomic DNA
ttggcataaaggacccgtaggcttaagtatatacattatatataatgtgtatttaataagatagcccgtcctccagaggaggccgggccatcctcgatagaatctattagctggtagtatgta

SEQ ID NO: 31 - DNA, 121 bp, Candida dubliniensis
  source 1..121: mol_type = genomic DNA
gctttagcagcaataatgtgttcaactaaagatctcttgtgtttaaaagtgttacctttggcagctttgtacaagttgtggtacaagtgtttatcaattttaccagcatctctgtatttag

SEQ ID NO: 32 - DNA, 121 bp, Candida tropicalis
  source 1..121: mol_type = genomic DNA
gctttagcagcaataatgtgttcaactaaagatctcttgtgtttgaaagtgttacctttagcagctttgtacaagttgtggtataagtgtttgtcaattttaccagcatctctgtatttag

SEQ ID NO: 33 - DNA, 121 bp, Candida parapsilosis
  source 1..121: mol_type = genomic DNA
gccttagcagtgatgatgtgttcaaccaaggatctcttgtgtttaaaggtgttacctttagcagccttgtacaagttgtggtacaagtgtctgtcaatcttaccagcatctctgtatttag

SEQ ID NO: 34 - DNA, 139 bp, Candida glabrata
  source 1..139: mol_type = genomic DNA
gaggccgactacacgacgacgatcgacaagggcaacggtgacttcgagacagaccttgtttcgcacatcacgaccaaggactctgacggcaagcctaccacgatcaccaccaccatcccattgaagccaagtgatgccg

SEQ ID NO: 35 - DNA, 106 bp, Candida krusei
  source 1..106: mol_type = genomic DNA
ggacattcagataatccagatttttcaaagatgagaaaaatttctaaaacatcgaatcccttacccgtgaagaaatatagtggacaaatatcagctaataaaatga

SEQ ID NO: 36 - DNA, 146 bp, Trichomonas vaginalis
  source 1..146: mol_type = genomic DNA
gtaacaaccttggagttcttcttgagctgaacacggacagccttacgaatagcagagtttggctgcttagcttcgacggcgaccttgccgacgacaatgccagatgccattgatgtaccttcgagtggtgtcttgtagattgatgt